(19) United States
(12) Patent Application Publication
(10) Pub. No.: US 2026/0273246 A1
(43) Pub. Date: Sep. 17, 2026

(54) FLUID CATHETER DEVICE FOR RECORDING BRAIN STATE

(71) Applicant: Biogen MA Inc., Cambridge, MA (US)

(72) Inventors: Daniel J. Abrams, Denver, CO (US); Matias Maturana, Melbourne (AU)

(73) Assignee: Biogen MA Inc., Cambridge, MA (US)

(21) Appl. No.: 19/677,198

(22) Filed: May 14, 2026

Related U.S. Application Data
(63) Continuation of application No. 17/380,415, filed on Jul. 20, 2021, now Pat. No. 12,685,851.
(60) Provisional application No. 63/053,864, filed on Jul. 20, 2020.

(51) Int. Cl. A61M 27/00; A61B 5/00; A61B 5/293; A61M 25/00; A61M 39/02

(52) U.S. Cl. CPC A61M 27/006; A61B 5/293; A61B 5/6852; A61B 5/6864; A61B 5/6868; A61M 25/0026; A61M 39/0247; A61M 2039/0273; A61M 2039/0276

(57) ABSTRACT

A device or system for delivering fluid to or removing fluid from a CSF-containing space of a brain and for recording electrical activity from white or grey matter in the brain includes a catheter including a proximal end, a distal end portion, a first lumen extending from the proximal end to the distal end portion, and one or more electrodes positioned relative to the catheter a distance from a distal end of the catheter, such that the one or more electrodes would be placed in contact with white or grey matter of the brain if the distal end of the catheter were positioned in the CSF-containing space. The catheter may include the electrodes or a lead adjacent the catheter may include the electrodes.

### RELATED APPLICATION

This application claims the benefit of U.S. Provisional Application No. 63/053,864 filed on Jul. 20, 2020, which application is hereby incorporated herein by reference in its entirety to the extent that it does not conflict with the disclosure presented herein.

### FIELD

The present disclosure relates to, among other things, devices and systems configured to carry fluid to or from a cerebrospinal fluid containing brain compartment and to record electrical signals from the brain.

### INTRODUCTION

Catheters and associated devices have been employed to deliver therapeutic agents to compartments of the brain comprising cerebral spinal fluid (CSF) or for withdrawing CSF from the brain. The catheters may be used to drain CSF from the brain, such as with a ventriculoperitoneal shunt or external ventricular drain; to percutaneously deliver therapeutic fluid to, or withdraw CSF from, the CSF space, such as with Ommaya or Rickman's reservoir; or to infuse therapeutic agent to the CSF space, such as with an implantable infusion device. Surgical procedures to implant the catheters and associated devices are invasive. For example, surgical placement of the catheter alone requires a burr hole to be drilled through the skull and the catheter to be advanced through brain tissue to reach the CSF-containing compartment.

In some cases, such catheters and associated devices may be used to monitor a patient's health or therapy progression. For example, if the device includes an access port for withdrawing CSF through catheter, the withdrawn CSF may be evaluated, for example, to determine whether the patient has a bacterial, fungal, or viral infection or to determine whether a delivered therapeutic agent is present in the CSF at effective concentrations. However, monitoring of electrical brain signals, which may be important for monitoring the therapy, a condition being treated, or a brain state, is not possible with such devices.

Brain activity may be monitored in such patients by using a separate system to record brain activity from the scalp. Such electroencephalogram (EEG) recordings may be useful for periodic monitoring of the patient's brain activity but are not suitable for long-term, continuous monitoring for patients that are not confined to a health care facility. In addition, the signal provided by such EEG recordings tends to be noisy and of lower quality due to signal attenuation through the skull and scalp.

### SUMMARY

The present disclosure relates to, among other things, devices and systems that include catheters for delivering therapeutic agents to the brain or withdrawing CSF from the brain and that include electrodes configured to be positioned within or close to white matter of the brain or grey matter of the brain. The electrodes may record electrical signals from within the brain.

By placing the electrodes within or in proximity to brain tissue, such as white matter or grey matter, less “noisy” signals may be obtained than with scalp-based EEG recordings. Such higher quality signals may facilitate interpretation of data recorded by the electrodes.

An electrode may be fixed a relative to a distal end of the catheter such that implanting the distal end of the catheter in the CSF-containing space causes the electrode to be placed in contact with the white or grey matter. The catheter may comprise the electrode. A separate lead may comprise the electrode. The lead may be placed adjacent to the catheter. If a separate lead comprises the electrode, the lead may be fixed relative to the catheter such that the electrode is a suitable distance from the distal end of the catheter to be positioned in contact with the white or grey matter when the distal end of the catheter is positioned in the CSF-containing space. Accordingly, a single implantation procedure may be performed to implant both the catheter and the electrode. Advantageously, the surgical implantation procedure is less complex and less invasive than procedures in which a catheter and a lead are separately implanted and positioned.

Preferably, the catheter comprises multiple lumens. A first lumen may be used, for example, to withdraw CSF from the brain, and a second lumen may be used, for example, to deliver a therapeutic fluid to the brain. The withdrawn CSF may be used to monitor disease or therapy progression, as well as monitor brain state, which may be used to, for example, adjust the rate of delivery of the therapeutic agent.

In addition or alternatively, data recorded by the electrode may be used to monitor disease or therapy progression, as well as monitor a current brain state or predict a future brain state. The rate of delivery of the therapeutic fluid may be adjusted based on data recorded by the electrode.

The combination of analysis of withdrawn CSF and data recorded by the electrode may result in substantially improved therapy and therapeutic outcomes than analysis of either alone.

According to an aspect of the present disclosure, a device or system for delivering fluid to or removing fluid from a CSF-containing space of a brain and for recording electrical activity from within the brain, comprises a catheter comprising a proximal end, a distal end portion, and a first lumen extending from the proximal end to the distal end portion. The device or system comprises an electrode positioned a distance from a distal end of the catheter such that the electrode would be placed in contact with white matter or grey matter of the brain if the distal end of the catheter were positioned in the CSF-containing space. If a distal end of the catheter is configured to be positioned in a cerebral ventricle, the electrode may be placed, for example, from about 0.5 centimeters to about 6 centimeters from the distal end.

The catheter may comprise the electrode. A lead may comprise the electrode. The lead may be secured relative to the catheter such that implanting the catheter causes the lead to be implanted such that when the distal end of the catheter is positioned in a cerebrospinal fluid (CSF)-containing space of a brain of a subject, the electrode of the lead is placed in contact with the white matter or the grey matter.

The device or system may comprise an access port. The access port may be configured to be implanted under the scalp of the subject. The access port may comprise an opening, a first catheter connector, and a first fluid flow path from the opening to the first catheter connector. The proximal end of the catheter may be coupled to the first catheter connector such that the first lumen is in communication with the first fluid flow path. The opening may be accessible by a needle inserted through skin of the subject, such as through the scalp of the subject, to allow delivery of fluid to the CSF-containing space or withdrawal of CSF through the first fluid flow path and first lumen.

The catheter may comprise a second lumen extending from the proximal end to the distal end portion. The access port may comprise a second fluid flow path extending from a second catheter connector to the first catheter connector. A device, such as an implantable infusion device, a CSF drainage catheter, or the like, may be operably coupled to the second catheter connector to allow therapeutic fluid to be infused through the second fluid flow path and second lumen or to allow CSF to drain from the CSF-containing space through the second lumen and second fluid flow path. Such a configuration permits CSF to be aspirated through the first flow path for analysis, while therapeutic fluid flow or CSF drainage continues through the second flow path.

The second fluid flow path may comprise a filter, such as a microbial filter. The first fluid flow path preferably does not comprise a microbial filter.

The device or system may comprise signal apparatus operably coupled to the one or more electrodes. The signal apparatus may be implanted in the subject and may be configured to process, transmit, or process and transmit data regarding the electrical signal recorded by the electrode.

The device or system may comprise an external apparatus configured to receive the data regarding the electrical signal transmitted by the signal apparatus. The external apparatus may be configured to wirelessly power the signal apparatus.

The device or system may be configured to alter the rate of delivery of therapeutic fluid based on the data regarding the electrical signal recorded by the electrode.

According to an aspect of the present disclosure, a device or system comprises an access port. The access port may be configured to be implanted under a scalp of a subject. The access port comprises (i) an opening accessible by a needle inserted through skin of the subject, such as the scalp of the subject, when the access port is implanted; (ii) a first catheter connector; (iii) a second catheter connector, (iv) a first fluid flow path extending from the opening to a first catheter connector; and (v) a second fluid flow path extending from the second catheter connector to the first catheter connector. The device or system further comprises a catheter coupled to, or operably couplable to, the first catheter connector. The catheter comprises a proximal end, a distal end portion, and first and second lumens extending from the proximal end to the distal end portion of the catheter. The first lumen is in communication with the first fluid flow path and the second lumen is in communication with the second fluid flow path when the proximal end of the catheter is coupled to the first catheter connector. The catheter has a length such that a distal end of the catheter is configured to extend to a CSF-containing space of a subject when the access port is implanted. The device or system also includes an electrode positioned a distance from a distal end of the catheter such that the electrode is positioned in white matter or grey matter of a brain of the subject when the access port is implanted and when the distal end of the catheter is positioned in the CSF-containing space.

The second fluid flow path may comprise a filter, such as a microbial filter. The first fluid flow path preferably does not comprise a microbial filter.

The catheter may comprise the electrode. A lead may comprise the electrode. The lead may be secured relative to the catheter such that implanting the catheter causes the lead to be implanted such that when the distal end of the catheter is positioned in the (CSF)-containing space of a brain of a subject, the electrode of the lead is placed in contact with the white matter or the grey matter.

The device or system may comprise signal apparatus electrically coupled to, or configured to electrically couple to, the electrode. The signal apparatus may be implanted in the subject and may be configured to process, transmit, or process and transmit data regarding the electrical signal recorded by the one or more electrodes.

The device or system may comprise an external apparatus configured to receive the data regarding the electrical signal transmitted by the signal apparatus. The external apparatus may be configured to wirelessly power the signal apparatus.

The device or system may be configured to alter the rate of delivery of therapeutic fluid based on the data regarding the electrical signal recorded by the electrode.

According to an aspect of the present disclosure, a method comprises recording an electrical signal from white matter or grey matter of a brain of a subject and determining a brain state of the subject or predicting a future brain state of the subject based on the recorded electrical signal.

Recording the electrical signal from the white matter or the grey matter of the brain of the subject may comprise recording the signal from an electrode positioned in the white matter or grey matter. A catheter may comprise the electrode. The catheter configured to deliver fluid to, or withdraw fluid from, a CSF-containing space of the subject. The catheter may be positioned to deliver fluid to, or withdraw fluid from, a CSF-containing space of the subject, such as a cerebral ventricle.

A lead may comprise the electrode. The lead may be fixed to a catheter configured to deliver fluid to, or withdraw fluid from, a CSF-containing space of the subject such that when the catheter is positioned to deliver fluid to, or withdraw fluid from, the CSF-containing space, the electrode of the lead is placed in contact with the white matter or the grey matter.

The method may further comprise applying an electrical signal to the white or grey matter. Recording the electrical signal from the white matter of the brain of the subject may comprise recording an electrical response evoked by the applied electrical signal. Applying an electrical signal to the white or grey matter may comprise applying the signal via a stimulating electrode positioned in the white or grey matter. The stimulating electrode may be the same as, or different than, the recording electrode.

The method may comprise provoking the subject. Recording the electrical signal from the white matter of the brain of the subject may comprise recording a provoked electrical response.

According to an aspect of the present disclosure, a method comprises delivering fluid to, or withdrawing fluid from, a CSF-containing space, such as a cerebral ventricle, of a brain of a subject. The fluid is delivered or withdrawn through a first lumen of a catheter. The method further comprises recording an electrical signal from the white or grey matter via an electrode. The electrode is positioned and fixed relative to the catheter such that implanting the catheter such that the catheter may deliver fluid to or with draw fluid from the CSF-containing space causes the electrode to be placed in contact with the white or grey matter.

The catheter may comprise the electrode. A lead may comprise the electrode. The lead may be fixed to a catheter configured to deliver fluid to, or withdraw fluid from, a CSF-containing space of the subject such that when the catheter is positioned to deliver fluid to, or withdraw fluid from, the CSF-containing space, the electrode of the lead is placed in contact with the white matter or the grey matter.

The method may comprise transmitting data regarding the recorded electrical signal to apparatus external to the subject.

The method may comprise determining a brain state of the subject or predicting a future brain state of the subject based on the recorded electrical signal.

The method may comprise applying an electrical signal to the white or grey matter. Recording the electrical signal from the white or grey matter of the brain of the subject may comprise recording an electrical response evoked by the applied electrical signal. The signal may be applied by the electrode positioned in white matter or grey of the brain.

According to an aspect of the present disclosure, a method comprises providing a device or system comprising an access port, a catheter, and an electrode. The access port may be configured to be implanted under a scalp of a subject. The access port comprises (i) an opening accessible by a needle inserted through skin of the subject, such as the scalp of the subject, when the access port is implanted; (ii) a first catheter connector; (iii) a second catheter connector; (iv) a first fluid flow path extending from the opening to a first catheter connector; and (v) a second fluid flow path extending from the second catheter connector to the first catheter connector. The catheter is coupled to, or operably couplable to, the first catheter connector. The catheter comprises a proximal end, a distal end portion, first and second lumens extending from the proximal end to the distal end portion of the catheter. The first lumen is in communication with the first fluid flow path and the second lumen is in communication with the second fluid flow path when the proximal end of the catheter is coupled to the first catheter connector. The catheter has a length such that a distal end of the catheter is configured to extend to a CSF-containing space of a subject when the access port is implanted in the subject. The electrode is positioned a distance from the distal end portion of the catheter such that the electrode is positioned in white or grey matter of a brain of the subject when the access port is implanted and the distal end of the catheter is positioned in the CSF-containing space. The method further comprises implanting the device or system such that the proximal end of the catheter is coupled to the first catheter connector, the first lumen of the catheter is in communication with the first fluid flow path of the access port, the second lumen of the catheter is in communication with the second fluid flow path of the access port, the distal end of the catheter is positioned in the CSF-containing space of the subject, and the electrode is positioned in the white or grey matter of the brain of the subject. The method also comprises recording an electrical signal from the white or grey matter via the electrode.

The second fluid flow path may comprise a filter, such as a microbial filter. The first fluid flow path preferably does not comprise a microbial filter.

The catheter may comprise the electrode. A lead may comprise the electrode. The lead may be fixed to the catheter such that when the distal end of the catheter is positioned in the CSF-containing space, the electrode of the lead is placed in contact with the white matter or the grey matter.

The method may comprise transmitting data regarding the recorded electrical signal to apparatus external to the subject. The method may comprise determining a brain state of the subject or predicting a future brain state of the subject based on the recorded electrical signal. The method may comprise applying an electrical signal to the white or grey matter, wherein recording the electrical signal from the white or grey matter of the brain of the subject comprises recording an electrical response evoked by the applied electrical signal. The signal may be applied by the one or more electrodes positioned in white or grey matter of the brain.

The method may comprise infusing a therapeutic fluid through the second fluid flow path of the access port and through the second lumen of the catheter. The second lumen may be in communication with the CSF-containing space, such as a cerebral ventricle. The method may comprise altering the rate at which the therapeutic fluid is infused based on data regarding the recorded electrical signal.

According to an aspect of the present disclosure, a method comprises implanting a catheter such that a distal end is positioned in a cerebrospinal fluid (CSF)-containing space of a brain of a subject. Implanting the catheter causes an electrode to be placed in contact with white matter or grey matter of the brain. The method includes infusing therapeutic fluid into the CSF-containing space, or withdrawing CSF from the CSF-containing space, via a lumen of the catheter. The method further includes recording an electrical signal from the white or grey matter via the electrode.

The catheter may comprise the electrode. A lead may comprise the electrode. The lead may be secured relative to the catheter such that implanting the catheter causes the lead to be implanted such that when the distal end of the catheter is positioned in a cerebrospinal fluid (CSF)-containing space of a brain of a subject, the electrode of the lead is placed in contact with the white matter or the grey matter.

The catheter may comprise multiple lumens that extend from a distal end portion to a proximal end portion. A first lumen may be configured for withdrawing CSF from the CSF-containing space in which the distal end of the catheter is implanted. A second lumen may be configured for introducing fluid into the CSF-containing space in which the distal end of the catheter is implanted.

The catheter may be a part of a system comprising an access port. The access port may comprise a first fluid pathway in fluid communication with the first lumen of the catheter and a second fluid pathway in fluid communication with the second lumen of the catheter. The second fluid pathway may comprise a filter, such as a microbial filter. Preferably, the first fluid pathway does not comprise a microbial filter.

The devices, systems, and methods described herein may be used for any suitable purpose, including treating a subject at risk or suffering from a brain disease.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic side view of an embodiment of catheter.

FIG. 2 is a schematic side view of an embodiment of catheter.

FIG. 3 is a schematic perspective view of an embodiment of a catheter.

FIG. 4 is a schematic perspective cut-away view of an embodiment of a portion of a catheter.

FIGS. 5-6 are schematic side views of embodiments of a lead coupled to a catheter via adhesive.

FIGS. 7A-B are schematic side views illustrating an embodiment of a lead, a catheter, and a sleeve configured to be (FIG. 7A) disposed about, or disposed about (FIG. 7B), the lead and the catheter.

FIG. 8 is a schematic side view illustrating an embodiment of a catheter comprising a groove configured to receive a lead.

FIG. 9 is a schematic top view of an embodiment of a connector configured to retain a lead relative to a catheter.

FIGS. 10A-B are schematic side views of an embodiment illustrating a lead, catheter, and connectors for securing the lead relative to the catheter.

FIG. 11 is a schematic view of an embodiment of implantable infusion device, signal apparatus, catheter with electrodes, and external apparatus.

FIG. 12 is a schematic view of an embodiment of an external apparatus being worn around an ear of a subject.

FIG. 13 is a schematic view of an embodiment of an implantable infusion device, a catheter containing electrodes and signal apparatus, and external apparatus.

FIG. 14 is a schematic view of an embodiment of an implantable infusion device containing signal apparatus, a catheter containing electrodes, and external apparatus.

FIG. 15 is a schematic side view of an embodiment of an access port.

FIG. 16 is a schematic bottom view of the embodiment of the access port depicted in FIG. 15.

FIG. 17 is a schematic top view of the embodiment of the access port depicted in FIGS. 15 and 16.

FIG. 18 is a schematic perspective view showing a bottom of an embodiment of an access port.

FIG. 19 is a schematic cross-sectional view of an embodiment of a catheter connector and interconnect.

FIG. 20 is a schematic bottom view of an embodiment of an access port having an interconnect.

FIG. 21 is a schematic side view of an embodiment of an access port having an interconnect.

FIG. 22 is a schematic top view of an embodiment of an access port having an interconnect.

FIG. 23 is a schematic side view of an embodiment of an access port having a passageway.

FIG. 24 is a schematic bottom view of an embodiment of an access port having a passageway.

FIG. 25 is a schematic sectional view illustrating an embodiment of an access port and a catheter implanted in a subject.

FIG. 26 is a schematic sectional view illustrating embodiment of an external ventricular drainage catheter and an intraparenchymal catheter implanted in a subject.

FIG. 27A and FIG. 27B show graphs illustrating evoked responses in the brain. FIG. 27A) Evoked responses were averaged over 30 seconds prior to (red) and after (blue) application of high frequency stimulation in the Anterior nucleus of the thalamus with parameters of 120 Hz, 2.5V and 300 microsecond duration. FIG. 27B) Evoked responses are used to detect the correct placement of DBS leads in Parkinson's. The evoked response seen in this figure is only observed in the subthalamic nucleus (STN) and disappears when the electrodes are placed in surrounding regions.

FIG. 28 are coronal and axial views of a catheter having a distal tip in a lateral ventricle and electrodes in white matter. Acute EEG recordings are shown on the right with numbering on electrodes corresponding to numbering on recording.

FIG. 29 is a schematic illustration of electrodes and signals in differential mode.

FIG. 30 is a schematic illustration of electrodes and signals in referential mode.

FIG. 31 is a schematic illustration of ground electrode placement that were evaluated.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components

### DETAILED DESCRIPTION

The present disclosure relates to, among other things, devices and systems that include catheters for delivering therapeutic agents to a cerebrospinal fluid (CSF)-containing space of a subject or withdrawing CSF from the CSF-containing space and that include an electrode configured to be positioned white or grey matter of the brain. The electrodes may record electrical signals from the white matter or the grey matter. Preferably the electrode records the electrical signals from the white matter.

The electrode may be positioned relative to the catheter such that implanting the catheter so that the catheter may deliver fluid to, or withdraw fluid from, the CSF-containing space causes the electrode to be positioned in the white matter or the grey matter of the brain of the subject. The catheter may comprise the electrode or a lead may comprise the electrode. The lead may be secured relative to the catheter such that implanting the catheter results in the electrode being positioned in the white or grey matter.

High quality electrical signals relating to brain activity may be obtained by electrodes positioned in the white or grey matter, as opposed to on an external surface of the scalp. White matter of the brain is composed mainly of long-range myelinated axons, and serves as a preferred target for monitoring general electrical activity of the brain. The electrodes in the white matter may capture electrical activity associated with general brain state rather than merely capturing signals localized to small regions of the brain. The high-quality brain activity signals recorded by electrodes in white matter may facilitate interpretation, processing, and analysis of the signals, which may be used for any suitable reason. Grey matter of the brain is composed mainly of neuronal cell bodies. The electrodes in grey matter may primarily capture local field potentials, or electrical activity associated with local regions of brain. Recording activity within confined brain regions, or interactions between two or more grey matter regions may facilitate interpretation, processing, and analysis of the signals, which may be used for any suitable reason.

Simply capturing the recorded electrical signals provides a substantial advantage relative to current therapies that deliver therapeutic fluid directly to the brain or withdraw CSF from the brain, because such current therapies do not provide a mechanism for capturing such electrical signals. The recorded electrical signals may provide meaningful insight into brain states of subjects suffering from diseases of the brain that are being treated with therapeutic fluid or CSF drainage. Such brain states are not very well understood. Capturing the recorded electrical signals in subjects receiving treatment via a catheter placed in the brain without additional substantial surgical complexity or invasiveness may provide meaningful advancement in the understanding of brain states in general and brain states of patients suffering from brain diseases in particular.

The recorded electrical signals may be used to understand a current brain state or to predict a future brain state. Preferably, the electrical signals may be employed to modify and improve the therapeutic fluid delivery therapy or CSF drainage therapy. For example, the rate of infusion of therapeutic fluid may be adjusted based on data regarding the recoded electrical signals.

The devices, systems, and methods described herein may be employed in any suitable subject suffering from a brain disease. Non-limiting examples of diseases for which placement of a catheter in a brain may be warranted include Parkinson's disease, Alzheimer's disease, dementia, Amyotrophic Lateral Sclerosis, Huntington's disease, lysosomal storage diseases, post-traumatic stress disorder, anxiety, depression, brain tumors, autism, autism spectrum disorder, traumatic brain injury, closed head injury, spinal cord injury, stroke, multiple sclerosis, schizophrenia, anxiety, and epilepsy.

Any catheter that may be placed into a CSF-containing space of a subject for delivering fluid to, or withdrawing fluid from, the CSF-containing space may be used or modified in accordance with the teachings presented herein. Examples of such catheters include catheters associated with implantable infusion devices, CSF shunt or drainage catheters, catheters associated with Ommaya reservoirs or Rickman reservoirs, catheters associated with any suitable access port, and the like.

A catheter as described herein includes a proximal end, a distal end, a distal end portion including and in proximity to the distal end, and at least one lumen extending from the proximal end to the distal end portion. Preferably, the distal end is placed in a CSF-containing space. For purposes of differentiation from other catheters that may be described herein, a catheter configured for positioning of a distal end in a CSF-containing space will be referred herein as a CSF catheter.

CSF exits the foramen of Magendie and Luschka to flow around the brainstem and cerebellum. CSF flows within the subarachnoid space. CSF is produced in the ventricular system of the brain and communicates freely with the subarachnoid space via the foramen of Magendie and Luschka. The distal end of the catheter may be placed anywhere that the CSF is accessible. For example, the distal end of the catheter may be placed in communication with a cisterna magna, a subarachnoid space, an intrathecal space, or a cerebral ventricle. Preferably, the distal end is placed in a cerebral ventricle. Preferably, the cerebral ventricle is a lateral cerebral ventricle.

The CSF catheter may be modified or adapted to include one or more recording electrodes as described herein. The one or more electrodes may be positioned on the CSF catheter a distance from the distal end such that the one or more electrodes would be placed in contact with white or grey matter of the brain if the distal end of the CSF catheter were at a suitable target location of the brain for therapeutic fluid delivery or CSF withdrawal (e.g., a CSF-containing space).

A lead that is separate from the CSF catheter may comprise the one or more electrodes. The lead may be coupled to the CSF catheter such that at least a portion of the lead is fixed relative to the CSF catheter. The lead may be fixed relative to the CSF catheter such that one or more electrodes of the lead may be positioned a distance from the distal end of the CSF catheter such that an electrode would be placed in contact with white or grey matter of the brain if the distal end of the CSF catheter were at a suitable target location of the brain for therapeutic fluid delivery or CSF withdrawal (e.g., a CSF containing space). For purposes of differentiation from other leads that may be described herein, a lead having an electrode configured to be positioned in white or grey matter of a brain of a subject will be referred herein as a brain lead.

The brain lead may be coupled to the catheter in any suitable manner. In some embodiments, the brain lead is coupled relative to the CSF catheter at the distal end portion of the CSF catheter, a mid-portion of the CSF catheter, and/or in proximity to the proximal end of the CSF catheter. Preferably, the brain lead is coupled relative to the CSF catheter at the distal end portion of the CSF catheter. Preferably, a distal portion of the brain lead is coupled to the distal end portion of the CSF catheter. By coupling a distal portion of the brain lead to a distal end portion of the CSF catheter, the distal portion of the brain lead may be pulled through brain tissue as the distal end portion of the CSF catheter is implanted and positioned in the CSF-containing space of the subject. Alternatively, a single implant procedure, e.g., implantation of the CSF catheter, results in implantation of both the CSF catheter and the brain lead.

In some embodiments, the brain lead is secured relative to the CSF catheter along a substantial length of the catheter or at multiple locations relative the CSF catheter. For example, the brain lead may be secured relative to the catheter over 50 percent or more of the length of the catheter, over 60 percent or more of the length of the catheter, over 70 percent or more of the length of the catheter, over 80 percent or more of the length of the catheter, or over 90 percent or more of the length of the catheter.

The brain lead may be secured relative to the catheter with adhesive, by bonding, via mechanical fixation, a combination thereof, or in any other suitable manner. In some embodiments, the brain lead is secured relative to the CSF catheter with a sleeve. The sleeve may be disposed over the brain lead and the CSF catheter. The sleeve may be elastomeric. The sleeve may be configured to press the brain lead against the CSF catheter when the sleeve is disposed over the CSF catheter. The sleeve may comprise windows configured to be positioned, or positioned, over the electrodes.

In some embodiments, a body of the CSF catheter defines an exterior groove configured to receive the brain lead. The groove may be configured to receive the brain lead via snap fit. In some embodiments, the CSF catheter comprises a groove and a sleeve, such an elastomeric sleeve, is placed about the brain lead and the CSF catheter to retain the brain lead in the groove of the CSF catheter.

A system or device as described herein may comprise a snap fit connector. The snap fit connector may engage one or both of the brain lead and the CSF catheter via snap fit engagement.

In some embodiments, the brain lead may be a currently available deep brain stimulation lead or may be a modified version of a deep brain stimulation lead. Currently available deep brain stimulation leads are available from Medtronic, Inc., Boston Scientific, Inc., and other manufacturers.

Preferably, the brain lead is coupled to the CSF catheter by a manufacturer. However, the brain lead may be coupled to the CSF catheter prior to implantation by, for example, a healthcare provider such as a surgeon. In such embodiments, the CSF catheter, the brain lead, or both the CSF catheter and the brain lead may comprise markings to facilitate proper alignment of the brain lead relative to the CSF catheter to ensure that, when the distal end of the CSF catheter is positioned in the CSF-containing space, the electrodes of the brain lead will be positioned in the white or grey matter.

The one or more electrodes, whether on the CSF catheter or a brain lead fixed relative to the CSF catheter, may be positioned any suitable distance from the distal end of the CSF catheter, depending in which CSF-containing space the distal end is placed. For example, at least one of the one or more electrodes may be positioned from about 0.5 centimeters to about 6 centimeters from a distal end of the CSF catheter, such as from about 1 centimeter to about 5 centimeters, or from about 2 centimeters to about 6 centimeters from the distal end of the CSF catheter.

The CSF catheter, or the brain lead that may be fixed relative to the CSF catheter, may comprise any suitable number of electrodes, such from about 1 electrode to about 64 electrodes. For example, the CSF catheter or the brain lead may contain 2 to 32 electrodes, such 2 to 16 electrodes, or 2 to 10 electrodes.

The electrodes may be longitudinally spaced relative to the CSF catheter at any suitable distance or interval. The electrodes may be evenly spaced or unevenly spaced. Preferably, at least some of the electrodes are evenly spaced. That is, the spacing between the electrodes relative to the length of the CSF catheter is preferably the same or substantially the same. As used herein, “substantially the same,” in the context of electrode spacing means that distances do not vary by more than 10%, preferably by not more than 5%.

The spacing between electrodes may vary depending on electrode width, number of electrodes, and desired distance that the electrodes span. The electrodes may span any suitable distance relative to the length of the CSF catheter. For example, the electrodes may span a length of about 5 centimeters, such as about 4 centimeters.

The electrodes may have any suitable width. The electrodes may have the same or different widths. Preferably, the electrodes have the same width. As an example, the electrodes may have a width of from about 0.5 millimeters to about 2 millimeters, such as from about 0.7 millimeters to about 1.5 millimeters.

In some embodiments, all the electrodes are positioned such that they will be placed in white or grey matter of the brain when implanted. However, it is not necessary that all the electrodes be placed in the white or grey matter. If an electrode is not placed in white or grey matter, the recording from that electrode may continue to be captured and potentially ultimately ignored. Alternatively, recording from that electrode may be inactivated.

Preferably, a majority of the electrodes are configured to be placed in white or grey matter when the catheter is implanted. Preferably, at least 70% of the electrodes are placed in white or grey matter when the catheter is implanted.

Preferably, at least two electrodes are configured to be placed in white or grey matter when the catheter is implanted. When multiple electrodes record signals from white or grey matter, coherent changes in activity between electrodes may be a powerful way to track more global changes. In some embodiments, the excitable state of a neural network is determined by monitoring a small neuronal population. The more excitable the small neuronal population, the higher the probability for activity to propagate throughout the network causing an ‘avalanche’ of activity. Such monitoring may be valuable for general brain state monitoring and may be particularly valuable for monitoring a brain state to predict a seizure.

The electrodes and associated signal processing apparatus may be configured in any suitable manner. For example, the electrodes and associated signal apparatus may be configured in differential mode or referential mode.

In differential mode, the system comprises an active electrode, a reference electrode, and a ground. The signal difference between an active electrode and a reference electrode may be amplified. The reference electrode may be a common reference for more than one active electrode. The reference electrode is preferably positioned a substantial distance from an active electrode and from the ground. The catheter or brain lead comprises the active electrode. The catheter or brain lead may comprise the reference electrode. The reference electrode may be separate from the catheter or brain lead. In differential mode, the system may be configured to detect small differences between electrode pairs and may be less likely to be affected by large artifacts originating near the ground electrode. However, the system may not be particularly effective at detecting larger common signals.

Preferably, the system is configured to detect larger common signals. Larger common signals may be associated with an overall brain state or with a seizure.

To detect larger common signals, the system may be configured in referential mode, which may also be referred to as single-ended mode. Referential mode may use a single active electrode per amplifier. There may be more than one active electrode. In referential mode, the output of the active electrode is amplified relative to the ground electrode, as opposed to the reference electrode in differential mode. The ground is preferably placed a substantial distance from the active electrode, which may result in amplification of signals that affect larger parts of the brain. While being effective at detecting larger common signals, referential mode may be sensitive to artifacts. Proper placement of the ground electrode may mitigate some issues associated with artifacts.

In referential mode, the catheter or the brain lead associated with the catheter comprises the active electrode. The catheter or the brain lead associated with the catheter may comprise the ground electrode. The ground electrode may be separate from the catheter or brain lead. If the catheter or associated brain lead comprises the ground electrode, the ground electrode may be positioned near the distal end of the catheter, near a mid-portion of the catheter, or near the proximal end of the catheter. Preferably, the ground electrode is placed near the proximal end of the catheter.

The electrodes may be positioned on the catheter or on the brain lead associated with the catheter in any suitable manner. For example, the electrodes may extend around the circumference of the catheter or brain lead or may extend less than all the way around the circumference of the catheter or brain lead. For example, the electrodes may radially extend around the catheter or brain lead from about 90 degrees to about 270 degrees, such as from about 120 degrees to about 240 degrees, from about 150 degrees to about 210 degrees, or about 180 degrees. Each electrode may extend around the catheter or brain lead the same or a different amount. Preferably, each electrode extends around the catheter, or the brain lead, the same amount. Preferably, each electrode extends radially around the catheter, or the brain lead, about 180 degrees, or about half-way around the circumference of the catheter or the brain lead. In some embodiments, every other electrode along the length of the catheter or the brain lead faces in substantially the same direction. Each subsequent electrode may face a substantially opposite direction as the adjacent electrode.

The electrodes may be made of any suitable material. Suitable materials for implantable electrodes are well-known to those of skill in the art. Materials suitable for deep brain stimulation electrodes are suitable materials for electrodes of the catheters described herein. In some embodiments, the electrodes are made from platinum or a platinum iridium alloy.

The electrodes may have any suitable thickness. For example, the electrodes may have a thickness from about 100 microns to about 0.3 millimeters, such as from about 200 microns to about 0.2 millimeters. The electrodes may be formed from a foil.

Each electrode may be discretely electrically coupled to one or more electrical contacts. An electrical interconnect may comprise the contacts. For purposes of differentiating from other electrical interconnects described herein, an electrical interconnect that comprises a contact that is electrically coupled with an electrode configured to be placed in white or grey matter of a subject's brain is referred to herein as a brain signal electrical interconnect.

The brain signal electrical interconnect may allow for electrical connection with another device or cable for connection to another device. In some embodiments, a proximal end portion of the catheter forms the electrical interconnect, with the contact being disposed on, or exposed through, an external surface of the catheter. In some embodiments, the electrical interconnect is physically separate from the catheter. In some embodiments, a proximal end portion of the brain lead forms the electrical interconnect, with the contact being disposed on, or exposed through, an external surface of the brain lead.

Regardless of the location of the brain signal electrical interconnect, conductors may electrically couple the electrodes to the contact. Each electrode may be electrically coupled to a discrete contact by a conductor. If the proximal end portion of the catheter serves as the electrical interconnect, the conductors may extend within a body of the catheter, within a lumen of the catheter, or along an external surface of the catheter. If the proximal end portion of the brain lead serves as the electrical interconnect, the conductors preferably extend within a body of the brain lead.

If the electrical interconnect is physically separate from the catheter (and the catheter comprises the electrodes), the conductors may run along a length of the catheter for a distance and branch off from the catheter as a cable through which the conductors extend to the electrical interconnect. The conductors may extend within a body of the catheter, within a lumen of the catheter, or along an external surface of the catheter before branching off from the catheter. The wires may be braided or twisted in the cable to improve flexibility, strength, or flexibility and strength.

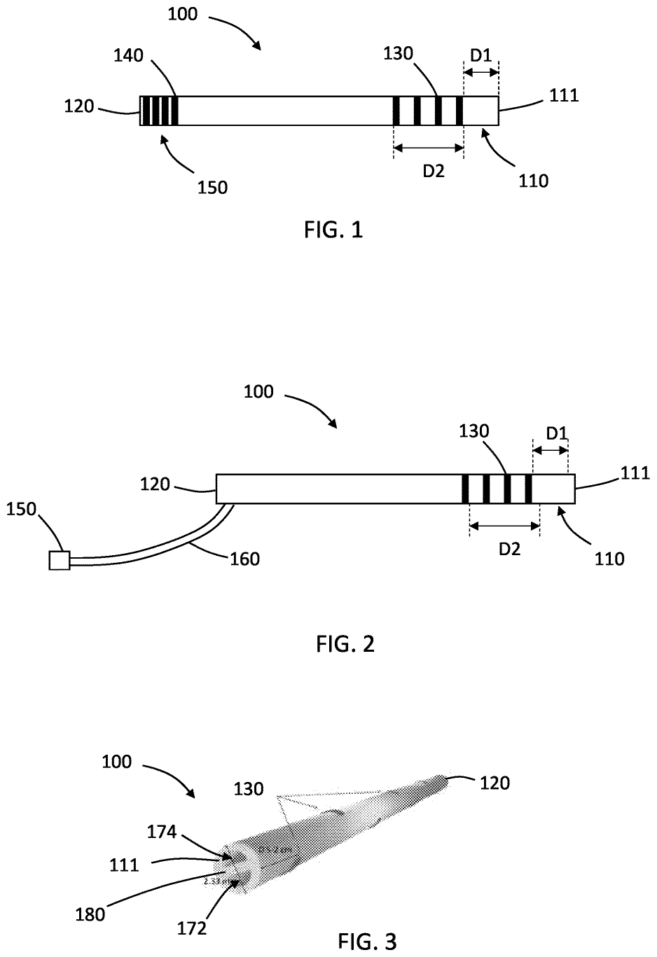

Referring now to FIGS. 1-4, embodiments of CSF catheters 100 are shown. The CSF catheters 100 have a proximal end 120 a distal end 111 at a distal end portion 110, and a lumen 172 extending from the proximal end 120 to the distal end 111. In some embodiments (not shown) the lumen 172 does not extend to the distal end 111, but rather the distal end portion 110 comprises openings (not shown) in communication with the lumen 172, 174 through which fluid may flow. The depicted CSF catheter 100 includes two lumens 172, 174, separated by a wall 180 that extends the length of the CSF catheter 100. However, the catheter 100 may have any suitable number of lumens.

The CSF catheter 100 has electrodes 130 positioned along a length of the catheter beginning at a distance D1 from the distal tip 111, such as about 0.5 centimeters to about 2 centimeters. The electrodes 130 may span a distance D2 along the length of the catheter 100, such as about 3 centimeters to about 5 centimeters.

In the embodiment depicted in FIG. 1, contacts 140 are positioned around the circumference of the CSF catheter 100 near the proximal end 120, forming an electrical interconnect 150.

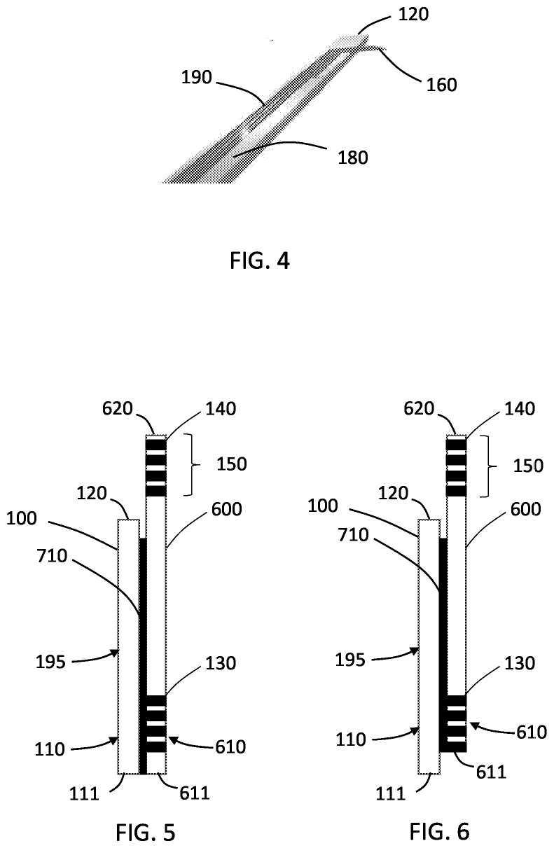

In the embodiment depicted in FIG. 2, the interconnect 150 is physically separate from the CSF catheter 100. A cable 160 containing conductors 190 (as shown in FIG. 4) extends to the interconnect 150.

The conductors 190 electrically couple the electrodes 130 to the contacts 140 at the interconnect 150. In FIG. 4, the conductors 190 are shown running along the wall 180 separating the lumens and branching off the CSF catheter as a cable 160. However, the conductors 190 may run along the CSF catheter at any suitable location, such as along an exterior surface.

Referring now to FIGS. 5 to 10B, embodiments that include a CSF catheter 100, a brain lead 600, and/or a fixation element or means are shown. The brain lead 600 may be placed adjacent to the CSF catheter 100. The brain lead 600 may be coupled to the CSF catheter 100 such that when the distal end 111 of the CSF catheter 100 is positioned in a CSF-containing space of a subject, one or more electrodes 130 of the brain lead 600 are in contact with white or grey matter of the brain of the subject.

Electrodes 130 are positioned at a distal end portion 610 of the brain lead 600. The distal end 611 of the brain lead 600 may be positioned adjacent the distal end 111 of the CSF catheter 100 (as shown in FIG. 5) or may be positioned proximal to the distal end 111 of the CSF catheter 100 (as shown in FIG. 6).

The brain lead 600 may be adhered to the CSF catheter 100 via adhesive 710 as shown in FIGS. 5-6. The adhesive 710 may be disposed in multiple locations along the CSF catheter 100 or may be disposed along a substantial portion of the CSF catheter 100 as shown in FIGS. 5-6. Preferably, the proximal end 120 of the CSF catheter 100 is not adhered to the brain lead 600 to facilitate connection to a catheter connector. This may also facilitate connection of the proximal end portion (in proximity to proximal end 620) of the lead to an interconnect to allow electrical connection between contacts 140 and a signal apparatus. The contacts 140 and body of the brain lead 600 may form an electrical interconnect 150 for electrically coupling to the signal apparatus interconnect or other suitable interconnect.

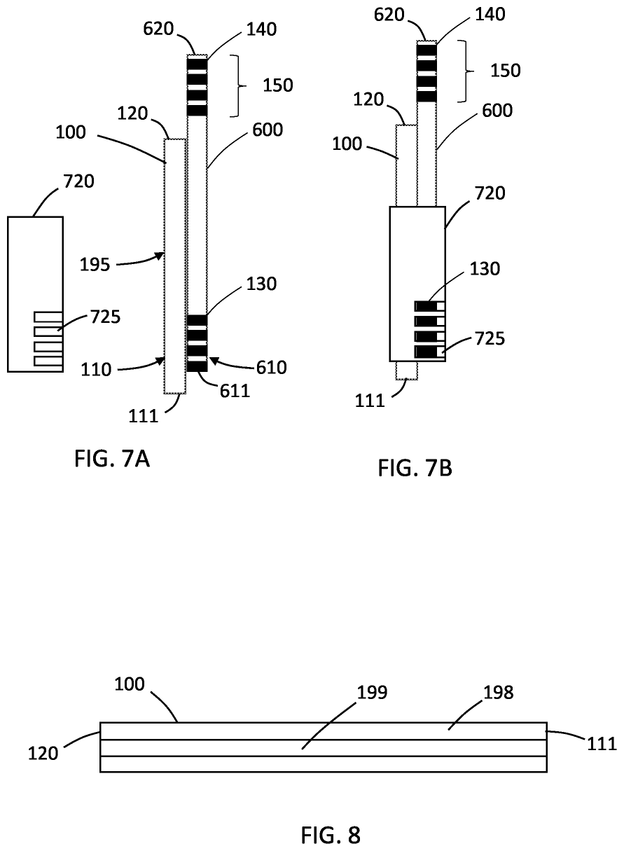

FIGS. 7A-B illustrated a CSF catheter 100, a brain lead 600, and a sleeve 720. The sleeve 720 may be disposed about the brain lead 600 and CSF catheter 100 and may be configured to press the brain lead 600 against the CSF catheter 100. Preferably, the sleeve 720 is elastomeric. The sleeve 720 may define windows configured to be placed over electrodes 130. The windows 720 (lack of material) allow the electrodes 130 to electrically couple with, or electrically contact, the white or grey matter in which they are implanted. That is, the electrodes 130 may be exposed to the tissue through the windows 725.

While only one sleeve 720 is depicted in FIGS. 7A-B, more than one sleeve may be employed. The sleeve 720 may be disposed over any suitable portion or substantially all of the brain lead 600 and CSF catheter 600.

FIG. 8 illustrates a CSF catheter 100 having a body 198 having an exterior surface defining a groove 199 configured to receive a brain lead (not shown in FIG. 8). The groove may extend the length of CSF catheter 100 (from the proximal end 120 to the distal end 111) or any suitable portion of the length of the CSF catheter 100. The groove 199 or one or more portions thereof may be configured to engage the brain lead via snap fit engagement. In some embodiments, the brain lead is received by the groove 199 and a sleeve (not shown in FIG. 8) is disposed about the brain lead and the CSF catheter 100 to retain the brain lead in the groove 199.

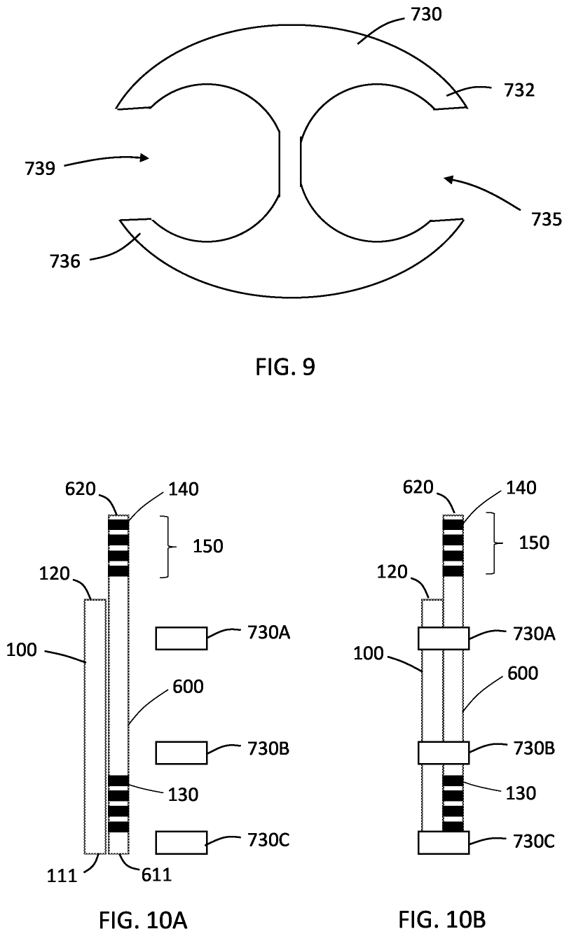

FIG. 9 shows an embodiment of a snap fit connector 730 configured to receive a CSF catheter and a brain lead by snap fit engagement. The connector 730 comprises an opening 735 configured to engage the CSF catheter and an opening 739 configured to engage the brain lead. The connector 730 comprises deflectable elements 732, 736 that are configured to resiliently deflect as the CSF catheter or brain lead are inserted into openings 735, 739 and thus engage the CSF catheter and brain lead via snap fit engagement.

FIGS. 10A-B show an embodiment in which snap fit connectors 730A-C, which may be a snap fit connector as shown in FIG. 9, are used to fix a brain lead 600 relative to a CSF catheter 100. One or more snap fit connectors 730A-C (three depicted) may be used to engage the CSF catheter 100 and the brain lead 600. The snap fit connectors 730 may be formed of any suitable material. Preferably, the snap fit connectors 730 comprise a biocompatible hard plastic material. Examples of suitable biocompatible hard plastic materials included polyether ether ketone, polycarbonate, polypropylene, and the like.

Other suitable connectors may be formed from biocompatible hard plastics or biocompatible metallic materials.

The devices, systems, or devices and systems describe herein may include signal apparatus electrically coupled to the electrodes. For example, the signal apparatus may be electrically coupled to the electrodes via an electrical interconnect of, for example, a cable extending from the CSF catheter, at the proximal end portion of a catheter, or the proximal end portion of a brain lead associated with the CSF catheter. The signal apparatus may process, transmit, or process and transmit data regarding the signals recorded by the electrodes. The signal apparatus may comprise any suitable components, such as components configured to one or more of: amplify, digitize, filter, and transmit data regarding the electrical signals recorded by the electrodes. For example, the signal apparatus may comprise one or more of: an amplifier, an analog to digital converter, a band pass filter, an antenna, and a transmission coil.

The signal apparatus may be configured to sample the signals from the electrodes at any suitable frequency. For example, the signal apparatus may be configured to sample the signals at a frequency of about 100 hertz or greater, such as 1,000 Hertz or greater. Preferably, the signal apparatus is configured to sample the signals at a frequency of about 10,000 hertz or greater. The signal apparatus may process the signal at any suitable bit depth, 4 bits, 8 bits, 16 bits or greater.

The signal apparatus may be implanted in the patient at any suitable location. The signal apparatus may comprise a power source or may be wirelessly powered. If the signal apparatus is wirelessly powered, the signal apparatus preferably includes an inductive coil, solenoid, or other suitable components to be wirelessly powered by an external apparatus and to transmit data regarding the signals recorded by the electrodes to the external apparatus. The signal apparatus is preferably implanted at a location where it may inductively couple with the apparatus external. For example, the signal apparatus may be positioned under the scalp of the subject near an ear of the subject. Such positioning may allow the external apparatus to be comfortably worn on or around the ear of the subject to provide suitable inductive coupling to power the signal apparatus and to wirelessly transmit data regarding the signals recorded by the electrodes from the signal apparatus to the external apparatus. The external device may then transfer the data to the cloud or to another device, such as a smart phone, a personal computer, or the like, which may then transfer the data to a server, or the like.

In some embodiments, the CSF catheter or the brain lead associated with the CSF catheter comprises the signal apparatus.

In some embodiments, an implantable infusion device comprises the signal apparatus.

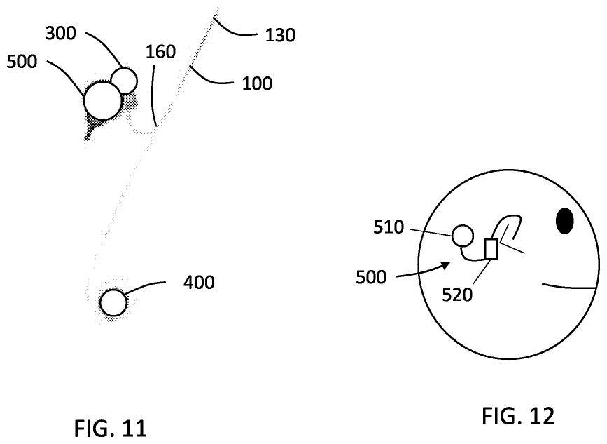

Referring now to FIGS. 11-14, various embodiments of CSF catheters 100 operably coupled to implantable infusion devices 400 are shown. In the depicted embodiments, the CSF catheters 100 comprise electrodes 130 for recording electrical signals in white matter. The electrodes 130 are electrically coupled to signal apparatus 300 configured to process, transmit, or process and transmit data regarding the electrical signals recorded by the electrodes 130. External apparatus 500 may be positioned relative to signal apparatus 300 such that the data may be transmitted wirelessly from the signal apparatus 30 to the external apparatus 500. The external apparatus 500 may be configured to wirelessly power the signal apparatus 300 in some embodiments.

For example and with reference to FIG. 11, the signal apparatus 300, which is coupled to electrodes 130 by conductors that run through cable 160 and through or along a portion of the CSF catheter 100, is implanted under the skin such that external apparatus 500 may wirelessly power and receive data from signal apparatus 300. For example and with reference to FIG. 12, the signal apparatus may be implanted under the subjects scalp near the ear. External apparatus 500 comprises an inductive coupling component 510 that may be positioned over the signal apparatus and comprises a processing component 520 operably coupled to the inductive coupling component 510. The processing component 520 may include, among other things, a rechargeable battery and a processor. The external apparatus 500 may transmit data received from signal apparatus 300, or a processed version thereof, to suitable secondary device, such as a smartphone, personal computer, tablet, modem, or the like through any suitable platform, such as low power Bluetooth. The secondary device may transmit data to the internet, where the data may be stored or retrieved by other computing devices as appropriate.

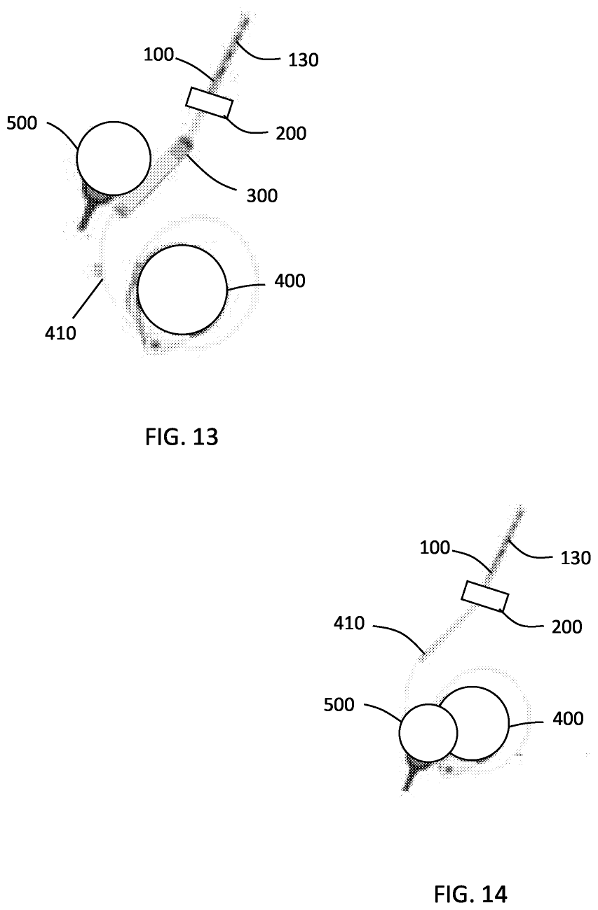

In the embodiment depicted in FIG. 13, an infusion catheter 410 extending from an implantable infusion device 400 to an access port 200 comprises the signal apparatus 300. The portion of the infusion catheter 410 comprising the signal apparatus 300 is preferably positioned at a location under the skin through which data regarding the signals recorded by the electrodes 130 may be transmitted to the external apparatus 500.

In the embodiment depicted in FIG. 14, the implantable infusion device 400 comprises the signal apparatus (not shown).

As indicated above, the CSF catheters described herein may be any suitable catheter, such as a catheter of an implantable infusion device, a catheter of a CSF shunt or drainage catheter, a catheter associated with an Ommaya or Rickman reservoir, or a catheter associated with an access port. For purposes of illustration, association of a CSF catheter with an access port will be described herein in more detail.

The devices, systems, or devices and systems described herein may include any suitable access port. The access port may be configured to be implanted below the scalp of the subject. The access port may comprise an opening configured to receive a needle inserted across the scalp of the subject. The access port may comprise a first catheter connector and a first fluid flow path extending from the opening to the first catheter connector. The proximal end of the catheter may be coupled to the first catheter connector such that the lumen of the catheter is placed in fluid communication with the first fluid flow path. The distal tip of the catheter may be implanted in a CSF-containing space of the brain of the subject such that the one or more electrodes are in the white matter.

A CSF catheter configured to couple to an access port implanted under the scalp may have any suitable length. Preferably, the length is sufficient to extend to a CSF-containing space, such as a cerebral ventricle. In some embodiments, the CSF catheter has a length from about 45 millimeters to about 80 millimeters. Preferably, the catheter has a length of about 60 millimeters to about 70 millimeters, more preferably from about 62 millimeters to about 68 millimeters within the cranium.

The needle may introduce fluid through a first fluid flow path of the access port and through the lumen of the catheter or may aspirate fluid from the CSF-containing space of the brain through the lumen of the catheter and the first fluid flow path.

At least a portion of the access port may be configured to be implanted in a burr hole in the skull of the subject, with a portion implanted above the skull.

The access port may comprise a second catheter connector and a second fluid flow path extending from the second catheter connector to the first catheter connector. In such embodiments, the CSF catheter may comprise a second lumen extending from the proximal end of the catheter to the distal end portion. When the proximal end of the CSF catheter is coupled to the first catheter connector, the second lumen may be placed in fluid communication with the second fluid flow path of the access port.

An implantable infusion device, a CSF drainage catheter, or the like may be operably coupled to the second fluid flow path of the access port via the second catheter connector. Any suitable implantable infusion device may be coupled to the second catheter connector. For example, the infusion device may be manually powered, electromechanically powered, chemically powered, or otherwise powered. In some examples, the infusion device may comprise a piston pump, a peristaltic pump, an osmotic pump, a plunger, or the like. Any suitable CSF drainage catheter may be coupled to the second catheter connector. For example, a distal portion of the CSF drainage catheter may be placed in a bladder or a peritoneal cavity or may extend external to the subject.

An access port having two isolated fluid flow paths as described above advantageously allows for therapeutic fluid to be continuously delivered, such as by an implantable infusion device, through the second fluid flow path while CSF is aspirated or another therapeutic fluid is delivered through the first fluid flow path. Similarly, if the second fluid flow path is used for CSF drainage, CSF may be aspirated, or a therapeutic fluid delivered, through the first fluid flow path without interrupting continuous CSF drainage through the second fluid flow path.

The second flow path may comprise a filter such as a microbial filter. A microbial filter may comprise a pore size of 0.45 microns or less, such as 0.22 microns or less or 0.2 microns or less. The first flow path preferably does not comprise a microbial filter. The second flow path may be used to sample CSF from the CSF-containing space of the subject. One purpose for sampling CSF is to determine whether an infection has occurred. If the first flow path comprises a microbial filter, an infectious microbe may be filtered (and not collected) and thus the presence of an infectious microbe may not be detected. In addition and over time, cells in the CSF and CSF proteins may accumulate on a microbial filter and may clog the filter. Accordingly, the first fluid flow path preferably does not comprise a microbial filter.

In embodiments where the cable carrying conductors that electrically couple the electrodes to the contacts of the interconnect branches off of the CSF catheter and the interconnect is physically separate from the CSF catheter or where a brain lead separate from the CSF catheter comprises the electrodes, the access port may define a passageway configured to receive the cable or lead. The passageway may extend from a bottom surface to a top surface or a side surface of the access port. In some embodiments, the passageway is of sufficiently large inner dimensions (e.g., width or diameter) to permit the interconnect of the cable or to permit the lead to be fed through the passageway. In some embodiments, the passageway may comprise a slot in an outer surface of the access port. The slot may be configured to receive the cable or lead.

In some embodiments, the access port comprises the electrical interconnect. For example, the electrical interconnect may be positioned at a top or side surface of the access port to facilitate connection with the signal apparatus or a lead extending to the signal apparatus. In such embodiments the CSF catheter, and brain lead if present, is preferably coupled to the access port by a manufacturer and the catheter, lead if present, and access port are constructed such that a portion of the cable or lead runs through the access port to the electrical interconnect.

In some embodiments, the access port comprises a first port interconnect configured to electrically couple to the electrical interconnect (e.g, of cable, proximal end portion of catheter, or proximal end portion of lead). The first port interconnect may be positioned at any suitable location of the access port, such as at a bottom surface of the access port. The access port may comprise a second port interconnect electrically coupled to the first port interconnect. The second port interconnect may be positioned at any suitable location of the access port. For example, the second port interconnect may be positioned at a top surface or side surface of the port or may extend from, or be tethered to, a top or side surface of the port.

The signal apparatus may be electrically coupled to the second port interconnect. For example, a signal apparatus lead having a lead interconnect may extend to the signal apparatus. The signal apparatus lead interconnect may be configured to electrically couple with the second port interconnect.

Additional details regarding suitable access ports (implantable cranial medical devices) and catheters that may be adapted for use herein are described in U.S. Provisional Patent Application No. 63/052,284, filed on 15 Jul. 2020, having a title of IMPLANTABLE CRANIAL MEDICAL DEVICE, naming Cerebral Therapeutics, Inc. as Applicant, and having Attorney Docket No. 0624.000008US60, which provisional patent application is hereby incorporated herein by reference in its entirety to the extent that it does not conflict with the disclosure presented herein.

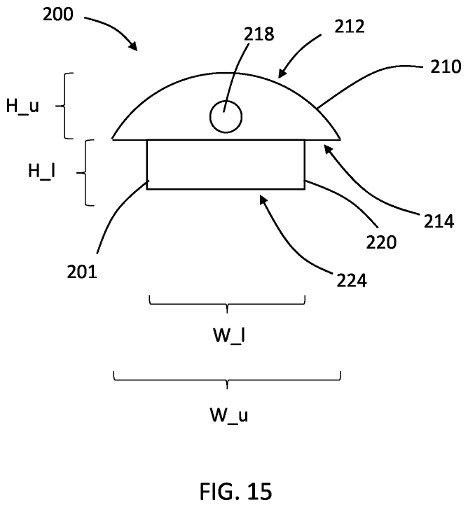

Reference is now made to FIGS. 15-17, which illustrate an example of an embodiment of an access port 200. The access port 200 comprises an upper flange portion 210 and a lower portion 220. The upper flange portion has a generally convex top surface 212 and a generally flat bottom surface 214. The upper flange portion 210 is configured to be positioned between a skull and a scalp of a subject when implanted, with the bottom surface 214 of the upper flange portion 210 configured to rest on a skull of a subject. The upper flange portion 210 has a height H_u and a width W_u. The height H_u of the upper flange portion 214 is sufficiently small to avoid skin erosion and substantial discomfort to the subject when implanted. The width W_u of the upper flange portion 214 is sufficiently large to rest on the skull around a burr hole.

The lower portion 220 has a bottom surface 224, a height H_I and a width W_I. The height H_I of the lower portion 220 is sufficiently small so that the bottom major surface 224 of the lower portion 220 does not extend substantially below the burr hole when implanted in a subject. Preferably, the bottom major surface 224 of lower portion 220 does not extend below the burr hole when implanted in a subject. The width W_I of the lower portion 220 is sufficiently small such that the lower portion 220 fits within the burr hole when implanted.

The upper flange portion 210 comprises a first opening 216 through which a needle may be inserted and a second catheter connector 218. The lower portion 220 has a first catheter connector defining a first opening 232 and a second opening 234. A first fluid flow path (not shown in FIGS. 15-17) extends within a housing 201 from the first opening 216 of the upper flange portion 210 to the first opening 226 of the lower portion 220. A second fluid flow path (not shown in FIGS. 15-17) extends within the housing 201 from the second catheter connector 218 of the upper flange portion 210 to the second opening 228 of the lower portion 220.

Reference is now made to FIG. 18, the first catheter connector 230 may include a slot 235 configured to receive a portion of a catheter that separates one lumen from another in a dual catheter. For example and with reference to FIG. 3, the wall 180 of the catheter 100 that separates the first lumen 172 from the second lumen 174 may be inserted into slot 235 of the first catheter connector 230 such that the first lumen 172 of the catheter 100 is placed in fluid communication with the first lumen 232 of the first catheter connector 230 and such that the second lumen 174 of the catheter 100 is placed in fluid communication with the second lumen 234 of the first catheter connector 230. FIG. 18 also illustrates an embodiment of a second catheter connector 218.

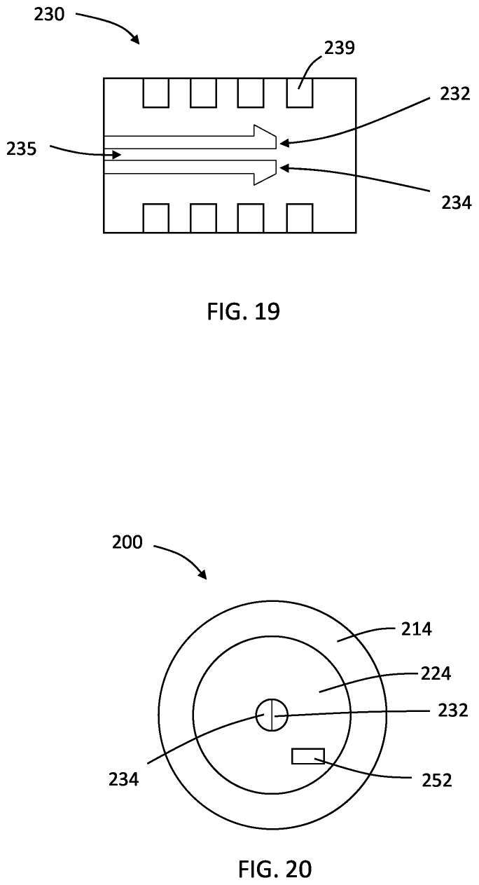

Referring now to FIG. 19, an alternative embodiment of a first catheter connector 230 is shown. As with the catheter connector 230 depicted in FIG. 18, the catheter connector 230 depicted in FIG. 19 includes a slot 230 separating extensions forming the first opening 232 and the second opening 234. The extensions comprise a barb feature configured to retain the catheter when inserted into the connector 230. The connector 230 also includes contacts 239 positioned within the receptacle such that when a catheter having proximal contacts is inserted into the receptacle to couple the catheter connector 230, the contacts 239 of the receptacle contact the proximal contacts of the catheter.

For example, if the proximal end 120 of the catheter 100 of FIG. 1 were inserted into the catheter connector of FIG. 19, the contacts 140 of the catheter 100 would contact and electrically couple with the contacts 239 of the receptable. Thus, the catheter connector 230 depicted in FIG. 19 may also serve as an electrical interconnect. In such an embodiment, the proximal portion of the catheter may be stiffened and/or reinforced to facilitate insertion into the catheter connector. It will be understood that the catheter connector 239 depicted in FIG. 19 may include sealing elements (not shown) to prevent bodily fluid from shorting or otherwise interfering with the electrical contact.

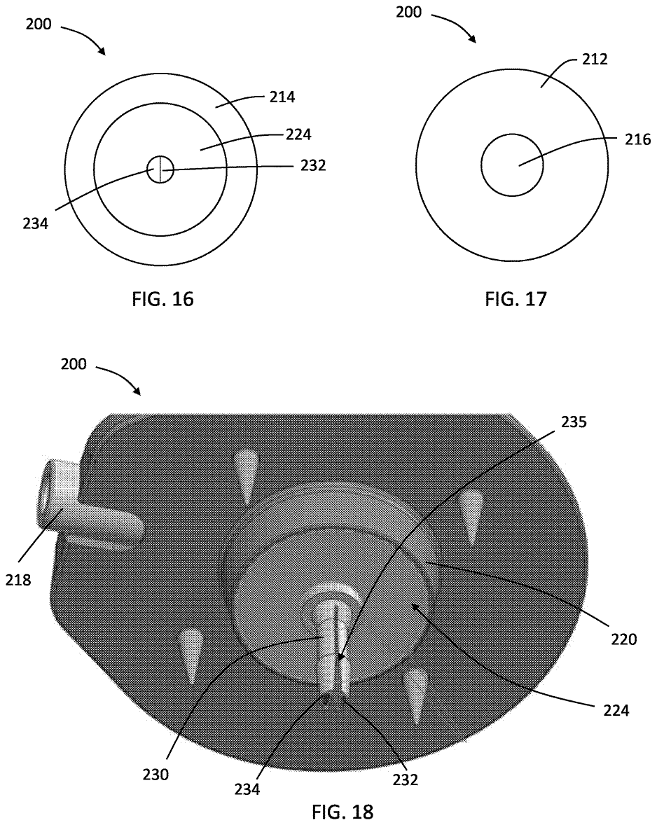

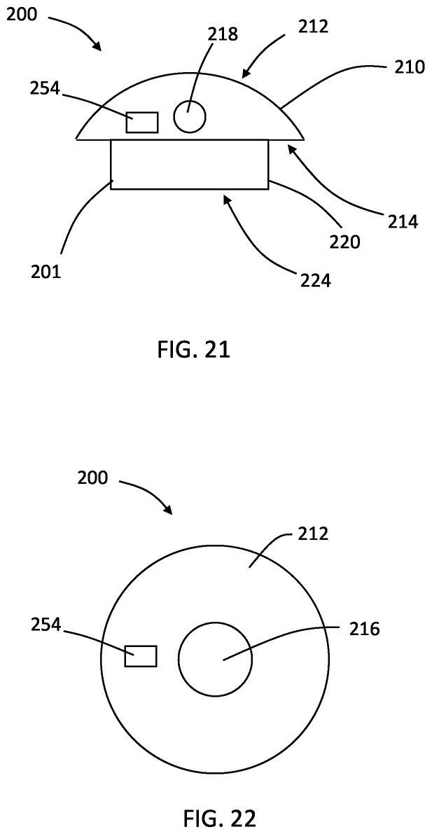

Referring now to FIGS. 20-22, embodiments of access ports 200 having electrical interconnects are shown. The embodiment depicted in FIG. 20 corresponds to the embodiment depicted in FIG. 16. The embodiment depicted in FIG. 21 corresponds to the embodiment depicted in FIG. 15. The embodiment depicted in FIG. 22 corresponds to the embodiment depicted in FIG. 17. To the extent that a labeled component is not discussed regarding FIGS. 20-22, reference is made to the discussion above regarding FIGS. 15-17.

As shown in FIG. 20, the access port 200 may comprise a first port electrical interconnect 252 at the bottom major surface 224 of the access port 200. The first port electrical interconnect 252 may be configured to connect with an interconnect that is physically separate from a catheter (e.g., a cable or a lead). For example, the proximal end 120 of the catheter 100 of FIG. 2 may be coupled to the catheter connector to place a first lumen of the catheter 100 in fluid communication with the first opening 232 of the access port 200 and to place a second lumen of the catheter 100 in fluid communication with the second opening 234 of the access port 200. The interconnect 150 of FIG. 2 may be connected with the first port interconnect 252.

The access port 200 may include a second port interconnect 254 at a side surface (FIG. 21) or the top surface (FIG. 22) of the access port 200. The second port interconnect 254 may be electrically coupled to the first port interconnect 252 through conductors (not shown) running in through the access port. The second port interconnect 254 may be connected with a lead interconnect of a lead that is operably coupled to signal apparatus to place the signal electronics in electrical communication with the electrodes of the catheter.

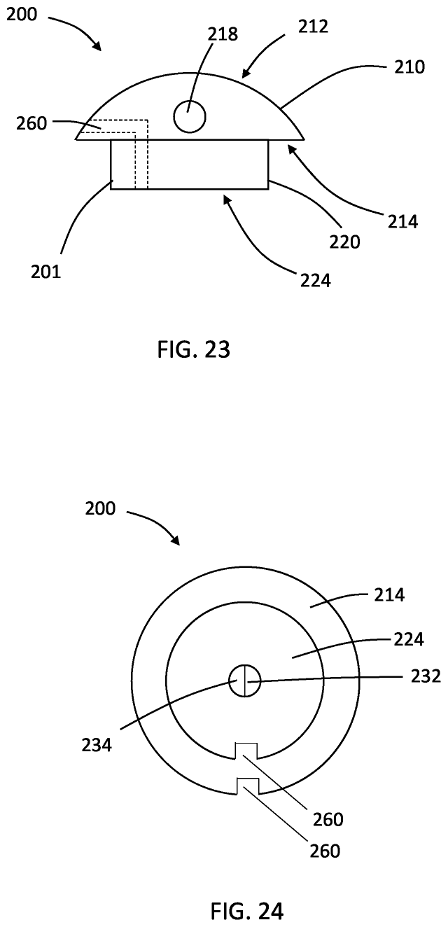

Referring now to FIGS. 23-24, access ports 200 having passageways 260 for receiving cables (e.g. cable 160 of FIG. 2 or a brain lead) are shown. The embodiment depicted in FIG. 23 corresponds to the embodiment depicted in FIG. 15. The embodiment depicted in FIG. 24 corresponds to the embodiment depicted in FIG. 16. To the extent that a labeled component is not discussed regarding FIGS. 23-24, reference is made to the discussion above regarding FIGS. 15-16.

In the embodiment depicted in FIG. 23, the passageway 260 extends from the bottom major surface 224 of the access port 200 to the top surface 212 and exits towards the side of the access port 200. The inner dimensions (e.g., width or diameter) of the passageway 260 may be sufficiently large to allow an interconnect (e.g., interconnect 150 of FIG. 2) or brain lead to be fed through the passageway 260. Alternatively, a manufacturer may construct the access port 200 with the CSF catheter coupled to first catheter connector (not shown in FIG. 17) and the cable or lead fed through the passageway 260 such that the interconnect extends from the passageway 260 to the side of the port 200.

In the embodiment depicted in FIG. 24, the passageway 260 is a slot formed in an outer surface of the access port 200. The slot may extend through the lower portion and the upper flange portion of the port 200. The cable and interconnect or lead may be guided to be received in the slot 260 as the access port 200 and catheter are being implanted.

In the embodiments depicted in FIGS. 23-24, the access port 200 permits the cable and interconnect or lead to be positioned above the skull and under the scalp so that the signal apparatus may be easily connected.

In some embodiments (not shown), the interconnect (e.g., interconnect 150 shown in FIG. 2 or proximal end portion of a brain lead) is incorporated into a top or side surface of the access port.

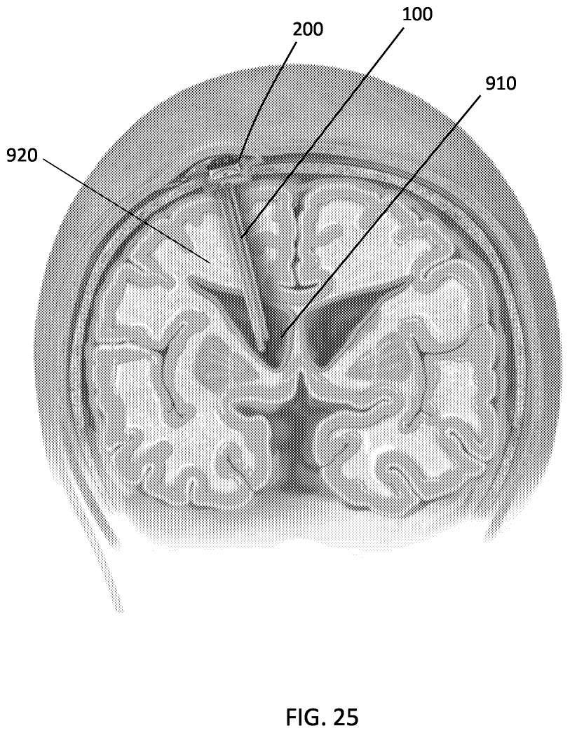

Referring now to FIG. 25, a catheter 100 and access port 200 are shown implanted in a patient. The access port 200 is implanted under the scalp. A portion of the access port 200 is above the skull and a portion is in a burr hole. The catheter 100 extends from the access port 200 to a lateral ventricle 910. Electrodes (not shown) of the catheter 100 (or of a lead coupled to the catheter, not shown) are positioned in the white matter or grey of the brain 920. Preferably, the electrodes are positioned in the white matter.

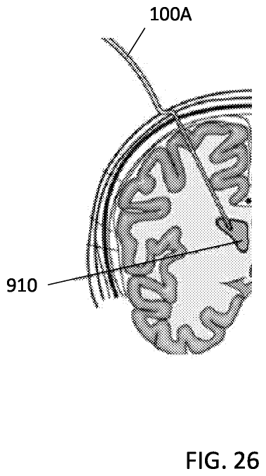

Referring now to FIG. 26, an external ventricular drainage cranial catheter 100A is shown with its distal end implanted in a lateral ventricle 910. Electrodes (not shown) of the catheter 100A (or of a lead coupled to the catheter, not shown) are positioned in the white matter or grey of the brain. Preferably, the electrodes are positioned in the white matter. The CSF drainage catheter may be a ventriculoperitoneal shunt or other suitable CSF shunt.

While of skill in the art will readily understand methods that may be employed to the devices and systems described herein. A brief description of some of the methods contemplated herein are described below.

In some embodiments, a method comprises recording an electrical signal from white or grey matter of a brain of a subject and determining a brain state of the subject or predicting a future brain state of the subject based on the recorded electrical signal. Recording the electrical signal from the white or grey matter of the brain of the subject may comprise recording the signal from one or more recording electrodes positioned in the white or grey matter. The recording electrodes may be on a catheter configured to deliver fluid to, or withdraw fluid from, a brain of the subject or may be on a lead coupled to the catheter. The catheter may be configured to deliver fluid to, or withdraw fluid from, a brain of the subject. The catheter may be positioned to deliver fluid to, or withdraw fluid from, a CSF-containing space of the subject. The catheter may be positioned to deliver fluid to, or withdraw fluid from, a cerebral ventricle of the subject.

The method may comprise applying an electrical signal to the white or grey matter. Recording the electrical signal from the white or grey matter of the brain of the subject may comprise recording an electrical response evoked by the applied electrical signal. Monitoring an evoked response may advantageously reduce relatively high intrinsic noise that may be present when passively monitoring, which noise may occlude a desired signal. Active monitoring (i.e., recording an evoked response) may improve the signal to noise ratio relative to passive monitoring. The electrical signal may be applied at regular intervals and the changes in evoked response may be recorded.

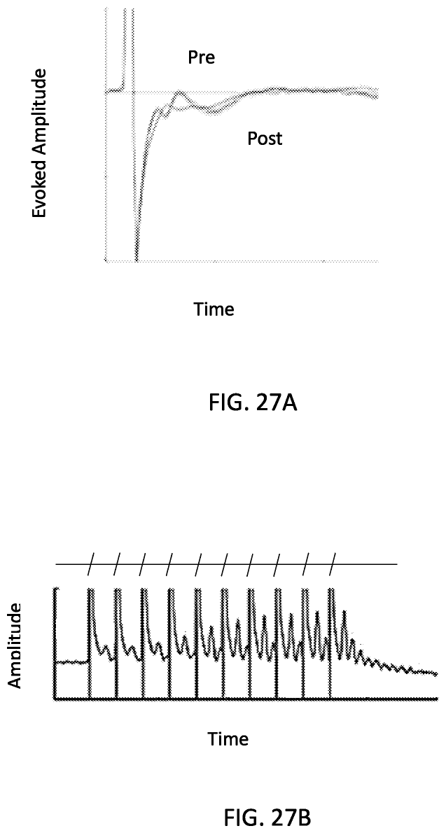

FIGS. 27A-B are provided to illustrate the use of evoked response to determine brain state. Active brain responses can be obtained by stimulating the brain at regular intervals and recording the changes in evoked brain response. The morphology of the evoked response could be used to determine properties about the neural network being stimulated. FIG. 27A shows a schematic example where 2 Hz electrical stimulation is applied to the hippocampus for 30 sec prior to (Pre) and following (Post) high frequency (120 Hz) stimulation applied to the anterior nucleus in a patient with epilepsy. The morphology of the evoked response had two clear bumps, forming a sinewave-like shape. Following the high frequency electrical stimulation, the response flattens out. This indicates that the high frequency stimulation interfered with the brain's response and changed its excitability

FIG. 27B shows another example where evoked responses are used to detect if an electrode has been correctly placed during implantation for Parkinson's disease. When stimulating the brain at 130 Hz, an evoked response is observed that increase in amplitude and then resonate at the termination of stimulation. This response is only observed when the electrodes are placed in the sub-thalamic nucleus and not in surrounding regions.

The method may comprise subjecting the subject to a provocation (“provoking the subject”) and measuring a response to the provocation. For purposes of this disclosure, a measuring a response to a provocation will be referred to as measuring a “provoked response.” A provocation may be a stimulus other than an electrical stimulus applied to the brain of a subject. A provocation may comprise, for example, a psychological or sensory stimulus associated with a disease from which the subject is at risk or is suffering. For example, the stimulus may be designed to treat or elicit a symptom of the disease. Examples of provocations include recall of a memory, an auditory stimulus, a visual stimulus, or a tactile stimulus.

As an example, a patient suffering from post-traumatic stress disorder (PTSD) may be asked to recall a traumatic memory, may be shown an image associated with a traumatic event, or the like. Electrical signals may be recorded by the one or more electrodes in the white or grey matter of the brain, and the provoked response may be monitored. The provoked response may be used to better understand changes in brain state or brain activity associated with the provocation. In some embodiments, delivery of therapeutic fluid through a lumen of the catheter may be altered based on the monitored provoked response.

As another example, a patient suffering from PTSD may undergo eye movement desensitization and reprocessing therapy (EMDR). EMDR is an interactive therapy in which a therapist directs the patient's eye movements while the patient is recalling traumatic event. Electrical signals may be recorded by the one or more electrodes in the white or grey matter of the brain, and the provoked response may be monitored. The provoked response may be used to better understand changes in brain state or brain activity associated with the provocation. In some embodiments, brain activity may be monitored when the subject recalls the traumatic event with or without concomitant eye movement. Insights may be gained regarding differences in brain state that may occur while the patient is undergoing EMDR relative to recalling a traumatic event. That is, EMDR may be an effective therapy for treating PTSD, but how such therapy affects the brain is not well understood. The methods and devices described herein may provide valuable insight into such therapies regardless of whether the therapies are directly associated with a therapy delivered through a lumen of a catheter as described herein.

As another example, a patient may be exposed to photic stimulation which may induce patients to have a seizure or manic electrophysiologic signature. The provoked response may be monitored, which may lead to better understanding neural links associated with such seizures and mania. Understanding these neural links can help develop therapies or use known therapies to modify progression of a manic prediction into mania and perform a rescue therapy.

In general, provocations may be used to identify electrical signals in the brain associated with the provoked response. Understanding such electrical signatures may facilitate improved therapy. By understanding the electrical signatures, brain activity events leading to such signatures may be determined, which may allow prediction of such events. By predicting such events, preventative therapy may be administered. Similarly, by understanding the nature of the signature electrical signal, and thus the ability to detect such signatures, recovery therapy may be administered more quickly following detection of a signature electrical signal.

In some embodiments, a method may comprise delivering fluid to, or withdrawing fluid from, a CSF-containing space of a brain of a subject, wherein the fluid is delivered or withdrawn through a first lumen of a catheter, the catheter comprising one or more electrodes positioned in white or grey matter of the brain or coupled to a lead comprising one or more electrodes positioned in white or grey matter of the brain. The method may also comprise recording an electrical signal from the white or grey matter via the one or more electrodes. The method may also comprise transmitting data regarding the recorded electrical signal to apparatus external to the subject. The method may comprise determining a brain state of the subject or predicting a future brain state of the subject based on the recorded electrical signal.

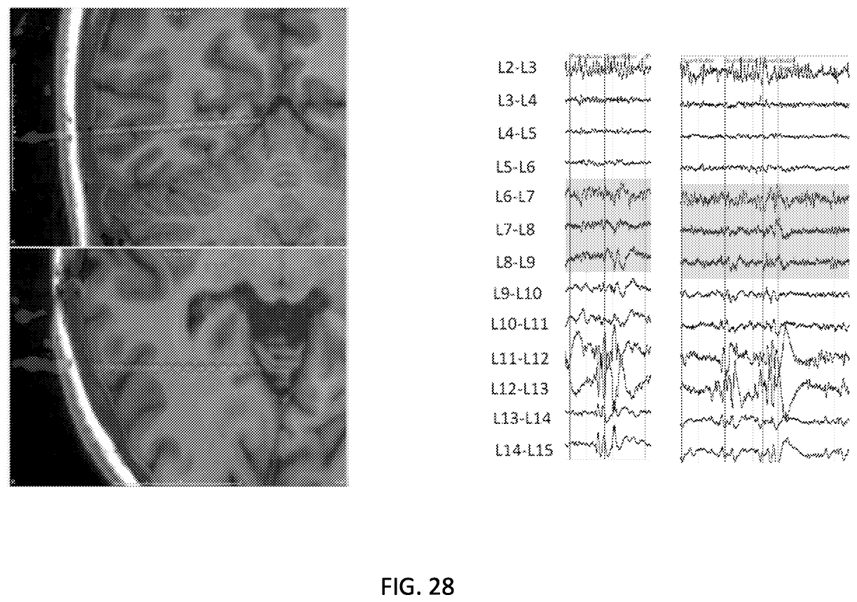

FIG. 28 shows an example of intracranial EEG recordings of the type that will be obtained by electrodes of the catheter. The left shows coronal and axial views of an electrode array in the white (L6-L9) and grey (L1-L5 and L10-L15) matter. Intracranial EEG recordings are shown on the right with numbering on electrodes corresponding to numbering on recording and the highlighted region showing white matter recordings. The signals represent changes in brain response as a subject performs a task (onset of task action is shown by red lines).

In some embodiments, a method includes monitoring the safety or effectiveness of the therapy delivered via a catheter or another therapy to which the subject is undergoing. Because the subject has a catheter comprising electrodes positioned to record electrical signals from white or grey matter implanted in their of the brain, the recorded electrical signals may be used to monitor any brain therapy that the subject is undergoing. Any procedure involving brain surgery presents risks. However, some procedures present more risk than others. For such risky procedures, monitoring safety may be particularly important. Safety may be monitored by monitoring electrical signals recorded by the one or more electrodes in the white or grey matter of the brain. For example, a recorded electrical signal having a signature associated with ischemia, necrosis, or other adverse event may be employed to alert a patient or health care provider to intervene. Preferably, the electrical signature may be detected before the subject presents with typically diagnosable symptoms, such as altered level of consciousness or focal neurological defect.

As an example, induced hypervolemia, hemodilution, and hypertension therapy (“triple H”) therapy may be effective but may present risks. Triple H therapy may be used to treat cerebral vasospasm after subarachnoid hemorrhage. Although Triple H therapy has gained widespread acceptance, some still question its efficacy and significant morbidity remains. Use of a catheter having electrodes, or coupled to a lead having electrodes, positioned in the white or grey matter as described herein may allow for monitoring of brain activity of a subject undergoing triple H therapy. The monitored activity may be used not only to determine whether triple H therapy is effective but also to determine if the therapy is safe. If brain signatures predictive of an adverse event are detected, medical intervention may occur.

In some embodiments, a method comprises delivering a therapeutic fluid to a central nervous system (CNS) of a subject suffering from or at risk of a CNS disease, and recording an electrical signal from white or grey matter of a brain of the subject. The therapeutic fluid may be delivered via a CSF catheter. The electrical signal may be recorded from an electrode of the CSF catheter or from an electrode of a brain lead coupled to the CSF catheter. The method may include developing a deep neural network (DNN) associated with the subject or the disease.

By collecting high quality data derived from within the brain (iEEG) and Artificial Intelligence (AI) techniques, an effective subject- or disease-specific DNNs may be obtained. The DNN may be used for any suitable purpose. For example, the DNN may be used for patient management. The DNN may support the construction of a non-invasive symptom management and diagnostic tool for non-implanted patients.

Deep neural networks (DNNs) are a sub-field of machine learning which leverage a composition of many nonlinear functions to map input data into a new desired output domain. The parameters of these nonlinear functions are not directly designed by humans, but instead learned from vast quantities of data. This allows the continual learning and improvement of a DNNs performance through the collection of more high-quality data. DNNs have found widespread success across numerous domains that often match or surpass human performance on specific tasks. Initially, deep learning strategies may be developed for iEEG analysis associated with waking EEG classification for emotions, motor activity, cognitive activity, seizure detection, and sleep scoring including convolutional neural networks and recurrent neural networks. The initial strategies may utilize one or both of supervised and unsupervised training approaches. The course of sleep disturbances may include ongoing analysis with local field potentials from deep brain electrodes subject to ongoing analysis by modern machine learning techniques as well as classical methods such as support vector machine and decision tree methods.

Suitable AI methods for establishing DNNs are described in U.S. Provisional Patent Application No. 63/054,522, entitled MONITORING AND TREATMENT BASED ON CONTINUOUS INTRACRANIAL EEG ACTIVITY, filed on Jul. 21, 2020, and naming Cerebral Therapeutics, Inc. as an Applicant, which is incorporated herein by reference in its entirety to the extent that it does not conflict with the disclosure presented herein.

### Use

Any suitable therapeutic fluid may be infused through a catheter described herein for treating a disease of the brain. The therapeutic fluid may comprise any suitable therapeutic agent. Preferably, the therapeutic agent is an agent for treating a disease of the brain. Examples of diseases of the brain include any diseases with pathology or dysfunction occurring in any component of the brain (including the cerebral hemispheres, diencephalon, brain stem, and cerebellum) or the spinal cord. Preferably, the devices and systems described herein are used to treat a disease of the brain that is resistant to treatment through systemic routes of administration, such as oral, intravenous, intramuscular, and intraperitoneal administration. The devices and systems described herein may also be used if a patient is at serious risk if direct central administration of a therapeutic fluid is not commenced.

The therapeutic fluid may be infused into the CSF or other brain region at any suitable rate. Preferably, flow rate into the brain is limited to 20 milliliters or less per day, such as 10 milliliters or less per day, or 5 milliliters per day or less. For example, the therapeutic fluid may be infused at a metered rate of 4 milliliters per day or less, such as 3 milliliters per day or less, 2 milliliters per day or less, or about 1 milliliter per day.

Withdrawal of CSF from the brain should be limited to 3,500 milliliters per day or less, preferably 200 milliliters per day or less.

The therapeutic fluid may comprise any suitable therapeutic agent. The therapeutic agent selected may depend on the disease being treated. The therapeutic fluid, such as a solution, may contain any suitable concentration of the therapeutic agent. The concentration of the therapeutic agent will depend on the therapeutic agent employed. In some embodiments, the therapeutic fluid is a solution comprising a therapeutic agent at a concentration in a range of from about 10 milligrams per milliliter to about 500 milligrams per milliliter, such as from about 50 milligrams per milliliter to about 450 milligrams per milliliter.

For purposes of illustration, a list of suitable anti-epileptic therapeutic agent that may be included in a therapeutic fluid, such as a solution, includes carbamazepine; tiagabine, levetiracetam; lamotrigine; pregabalin; fenfluramine; gabapentin; phenytoin; topiramate; oxcarbazepine; valproate; valproic acid; zonisamide; perampanel; eslicarbazepine acetate; lacosamide; vigabatrin; rufinamide; fosphenytoin; ethosuximide; phenobarbital; diazepam; lorazepam; clonazepam; clobazam; ezogabine; felbamate; primidone; acetazolamide; brivaracetam; clorazepate; ethotoin; mephenytoin; methsuximide; trimethadione; bumetanide; adenosine; and an adenosine al receptor agonist. In some embodiment, the therapeutic agent is valproic acid or a pharmacologically acceptable salt thereof. For purposes of the present disclosure, reference to a compound includes reference to salts, hydrates, solvates, and polymorphs thereof.

Examples of other therapeutic agents that may be included in a therapeutic fluid for treating or diagnosing a CNS disease include Edaravone (e.g., Radicava®) for Amyotrophic Lateral Sclerosis (ALS), Valbenazine (e.g., Ingrezza®) for Tardive dyskinesia, Deuterabenazine (e.g., Austedo®) for Huntington's disease, Ocrelizumab (e.g., Ocrevus®) for Multiple sclerosis, Safinamide (e.g., Xadago®) for Parkinson's disease, Nusinersen (e.g., Spinraza®) for Spinal muscular atrophy (SMA), Daclizumab (e.g., Zinbryta®) for Multiple sclerosis, Pivavanserin (e.g., Nuplazid®) for Hallucinations and delusions associated with psychosis, Ariprprazole lauroxil (e.g., Aristada®) for Schizophrenia, Caripazine (e.g., Vraylar®) for Schizophrenia and bipolar disorder, Brexpiprazole (e.g., Rexulti®) for Schizophrenia, Peginterferon beta-la (e.g., Plegridy®) for Multiple sclerosis, Eslicarbazepine acetate (Aptiom®) for Epilepsy associated seizures, Flutemetamol F 18 (e.g., Vizamyl®) Radioactive diagnostic for Alzheimer's disease, Vortioxetine (e.g., Brintellix®) for Major depressive disorder, Dimethyl fumerate (e.g., Tecfidera®) for multiple sclerosis, and Gadoterate megumine (e.g., Dotarem®) for MRI-based brain imaging.

Another example of therapeutic agents that may be included in a therapeutic fluid is a Dominant Negative Tumor Necrosis Factor (DN-TNF) such as XPRO®1595 and the like.

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

Any direction referred to herein, such as “top,” “bottom,” “side,” “upper,” “lower,” and other directions or orientations are described herein for clarity and brevity but are not intended to be limiting of an actual device or system. Devices and systems described herein may be used in a number of directions and orientations.

As used herein, “providing” a device or system means manufacturing the device or system, assembling the device or system, purchasing the device or system, or otherwise obtaining the device or system.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must).

The words “include,” “including,” and “includes” indicate open-ended relationships and therefore mean including, but not limited to. Similarly, the words “have,” “having,” and “has” also indicated open-ended relationships, and thus mean having, but not limited to. Similarly, the terms “comprise” and “comprising” indicate open-ended relationships, and thus mean comprising, but not limited to. The terms “consisting essentially of” and “consisting of” are subsumed within the term “comprising.” For example, a catheter comprising tubing may be a catheter consisting of tubing. The term “consisting essentially of” means a recited list of one or more items belonging to an article, kit, system, or method and other non-listed items that do not materially affect the properties of the article, kit, system, or method.

The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a catheter connector may be configured to place a lumen of a catheter in fluid communication with a fluid path, even when the catheter is not connected to the catheter connector).

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

The invention is defined in the claims. However, below there is provided a non-exhaustive list of non-limiting examples. Any one or more of the features of these examples may be combined with any one or more features of another example, embodiment, or aspect described herein.

Example Ex1 A device or system for delivering fluid to or removing fluid from a cerebrospinal fluid (CSF)-containing space of a brain and for recording electrical activity from white or grey matter in the brain, the device or system comprising: (i) a CSF catheter comprising a proximal end, a distal end portion, and a first lumen extending from the proximal end to the distal end portion; and (ii) an electrode positioned a distance from a distal end of the CSF catheter such that the electrode would be placed in contact with white or grey matter of the brain if the distal end of the CSF catheter were positioned in the CSF-containing space.

Example Ex2 The device or system of Ex1, wherein the electrode is positioned a distance from about 0.5 centimeters to about 6 centimeters to the distal end of the CSF catheter.

Example Ex3 The device or system of Ex2 wherein the electrode is positioned a distance from about 1 centimeter to about 5 centimeters to the distal end of the CSF catheter.

Example Ex4 The device or system of Ex1, wherein the electrode is positioned a distance from about 2 centimeters to about 6 centimeters to the distal end of the CSF catheter.

Example Ex5 A device or system for delivering fluid to or removing fluid from a cerebrospinal fluid (CSF)-containing space of a subject and for recording electrical activity from white or grey matter in a brain of the subject, the device or system comprising: (i) a CSF catheter comprising a proximal end, a distal end portion, and a first lumen extending from the proximal end portion to the distal end portion; and (ii) an electrode positioned relative to the CSF catheter at a distance from about 0.5 centimeters to about 6 centimeters to a distal end of the CSF catheter.

Example Ex6 A device or system according to Ex5, wherein the electrode is positioned from about 1 centimeter to about 5 centimeters from the distal end of the catheter.

Example Ex7 A device or system according to Ex5, wherein the electrode is positioned from about 2 centimeters to about 6 centimeters from the distal end of the catheter.

Example Ex8 The device or system of any one of Ex1 to Ex7, wherein the CSF catheter comprises the electrode.

Example Ex9 The device or system of any one of Ex1 to Ex7, further comprising a brain lead, wherein the brain lead comprises the electrode and wherein the brain lead is coupled to the CSF catheter.

Example Ex10 The device or system of Ex9, wherein the brain lead is coupled relative to the CSF catheter at the distal end portion of the CSF catheter.

Example Ex11 The device or system of Ex9 or Ex10, wherein the brain lead is coupled to the CSF catheter in proximity to a mid-portion of the CSF catheter.

Example Ex12 The device or system of any one of Ex9 to Ex11, wherein the brain lead is coupled to the CSF catheter in proximity to the proximal end of the CSF catheter.

Example Ex13 The device or system of any one of Ex9 to Ex12, wherein the brain lead is secured to the CSF catheter along a substantial length of the CSF catheter.

Example Ex14 The device or system of any one of Ex9 to Ex13, wherein the brain lead is secured to the CSF catheter with adhesive.

Example Ex15 The device or system of any one of Ex9 to Ex14, wherein the brain lead is mechanically fixed relative to the CSF catheter.

Example Ex 16 The device or system of any one of Ex9 to Ex15, comprising a sleeve disposed over the brain lead and the CSF catheter.

Example Ex17 The device or system of any one of Ex9 to Ex16, comprising a sleeve, wherein the sleeve is configured to be disposed over the brain lead and the CSF catheter.

Example Ex18 The device or system of Ex16 or Ex17, wherein the sleeve is elastomeric and configured to press the brain lead against the CSF catheter when the sleeve is disposed over the brain lead and the catheter.

Example Ex 19 The device or system of any one of Ex16 to Ex18, wherein the sleeve comprises a window positioned over, or configured to be positioned over, the electrode.

Example Ex20 The device or system of any one of Ex9 to Ex19, wherein the CSF catheter defines a groove and wherein the brain lead is received in the groove or is configured to be received in the groove.

Example Ex21 The device or system of Ex20, wherein the groove engages, or is configured to engage, the brain lead via snap fit.

Example Ex22 The device of system of any one of Ex9 to Ex21, further comprising a snap fit connector, wherein the CSF catheter and brain lead are engaged by, or are configured to be engaged by, the snap fit connector.

Example Ex23 The device or system of any one of Ex1 to Ex22, wherein the electrode would be placed in contact with the white or grey matter of the brain if the distal end of the CSF catheter were positioned in a lateral cerebral ventricle.

Example Ex24 The device or system of any one of Ex1 to Ex23, comprising one to sixty-three additional electrodes.

Example Ex25 The device or system of any one of Ex1 to Ex24, comprising a brain signal electrical interconnect, wherein the brain signal electrical interconnect comprises a contact electrically coupled to the electrode, and comprises one to sixty-three additional contacts each discretely electrically coupled to one of the one to sixty-three additional electrodes if present.

Example Ex26 The device or system of Ex25, wherein the CSF catheter comprises the brain signal electrical interconnect.

Example Ex27 The device or system of Ex25 comprising the brain lead according to claim aspect 9, wherein the brain lead comprises the brain signal electrical interconnect.

Example Ex28 The device or system of Ex27, wherein the brain signal electrical interconnect is in proximity to a proximal end of the brain lead.

Example Ex29 The device or system of Ex25, wherein the CSF catheter comprises the electrode and wherein the brain signal electrical interconnect is physically separate from the CSF catheter.

Example Ex30 The device or system of Ex29, further comprising a cable extending from the CSF catheter to the brain signal electrical interconnect, wherein the cable comprises a conductor electrically connecting the electrode to the contact.

Example Ex31 The device or system of any one of Ex1 to Ex30, further comprising an access port having a first fluid flow path in communication with the first lumen of the CSF catheter or having a connector configured to secure the CSF catheter such that first lumen is in communication with the first fluid flow path.

Example Ex32 The device or system of Ex31, wherein the access port is configured to be implanted below a scalp.

Example Ex33 The device or system of Ex32, wherein at least a portion of the access port is configured to be implanted over a burr hole.

Example Ex34 The device or system of Ex32 or Ex33, wherein at least a portion of the access port is configured to be implanted in the burr hole.

Example Ex35 The device or system of any one of Ex31 to Ex34, wherein the access port defines a passageway configured to receive the cable of the device or system according to claim 30.

Example Ex36 The device or system of any one of Ex31 to Ex34, comprising the brain lead, wherein the access port defines a passageway configured to receive the brain lead.

Example Ex37 The device or system of Ex35 or Ex36, wherein the passageway extends though the access port from a bottom surface to a top surface or to a side surface.

Example Ex38 The device or system of any one of Ex35 to Ex37, wherein passageway has a sufficiently large inner dimensions for the electrical interconnect to be fed through the passageway.

Example Ex39 The device or system of Ex38, wherein the passageway comprises a slot in an outer surface of the access port.

Example Ex40 The device or system of any one of Ex31 to Ex39, wherein the access port comprises a first port interconnect configured to electrically couple to the brain signal electrical interconnect of any one of Ex26 to Ex30.

Example Ex41 The device or system of Ex40, wherein the access port comprises a second port interconnect, wherein the second port interconnect is electrically coupled to the first port interconnect.

Example Ex42 The device or system of any one of Ex1 to Ex41, further comprising signal apparatus for processing, transmitting, or processing and transmitting data regarding the signals recorded by the electrode.

Example Ex43 The device or system of Ex42, wherein the signal apparatus is configured to be implanted in a patient.

Example Ex44 The device or system of Ex42 or Ex43, further comprising an implantable infusion device, wherein the implantable infusion device comprises the signal apparatus.

Example Ex45 The device or system of any one of Ex42 to Ex44, further comprising an external apparatus configured to receive transmitted data from the signal apparatus.

Example Ex46 The device or system of any one of Ex42 to Ex45, wherein the signal apparatus is electrically coupled to, or configured to be electrically coupled to, the second port interconnect according to claim 41 or the brain signal interconnect according to any one of Ex26 to Ex30.

Example Ex47 The device or system of Ex46, further comprising a signal apparatus lead comprising a signal apparatus lead interconnect, wherein the signal apparatus lead is electrically coupled to the signal apparatus.

Example Ex48 The device or system of Ex47, wherein the signal apparatus lead interconnect is configured to electrically couple with the electrical interconnect or the second port interconnect or the brain signal interconnect of any one of Ex23 to Ex29.

Example Ex49 The device or system of any one of Ex1 to Ex48, wherein the CSF catheter comprises a second lumen extending from the proximal end to the distal end portion.

Example Ex50 The device or system of Ex45, comprising an access port according to any one of Ex31 to Ex41, wherein the access port defines a second fluid flow path in fluid communication or configured to be placed in fluid communication with the second lumen of the CSF catheter.

Example Ex51 The device or system of Ex50, wherein the access port is configured to be operably coupled with an implantable infusion device such that therapeutic fluid from the implantable infusion device may be infused through the second fluid flow path and the second lumen of the CSF catheter.

Example Ex52 The device or system of Ex51, wherein the second fluid flow path comprises a microbial filter.

Example Ex 53 The device or system of Ex52, wherein the first fluid flow path is free from a microbial filter.

Example Ex54 The device or system of any one of Ex50 to Ex53, wherein the access port is configured to connect to a drainage catheter such that CSF from the CSF-containing space may be drained through the second lumen of the CSF catheter, the second fluid path of the access port, and through a drainage lumen of the drainage catheter.

Example Ex55 The device or system of any one of Ex1 to Ex54, wherein the CSF catheter has a length from about 45 millimeters to about 80 millimeters within the cranium.

Example Ex56 The device or system of any one of Ex1 to Ex54, wherein the CSF catheter has a length from about 60 millimeters to about 70 millimeters within the cranium.

Example Ex57 The device or system of any one of Ex1 to Ex54, wherein the CSF catheter has a length from about 62 millimeters to about 68 millimeters within the cranium.

Example Ex58 A method comprising: (i) recording an electrical signal from white or grey matter of a brain of a subject; and (ii) determining a brain state of the subject or predicting a future brain state of the subject based on the recorded electrical signal.

Example Ex59 The method of Ex58, wherein recording the electrical signal from the white or grey matter of the brain of the subject comprises recording the signal from an electrode positioned in the white or grey matter.

Example Ex60 The method of Ex59, wherein the electrode is on a catheter positioned to deliver fluid to, or withdraw fluid from, a brain of the subject.

Example Ex61 The method of Ex 59, wherein the catheter is positioned to deliver fluid to, or withdraw fluid from, a CSF-containing space of the subject.

Example Ex62 The method of Ex61, wherein the catheter is positioned to deliver fluid to, or withdraw fluid from, a cerebral ventricle of the subject.

Example Ex63 The method of Ex59, wherein the electrode is on a lead, and wherein the lead is coupled to a catheter.

Example Ex64 The method of Ex63, wherein the catheter is positioned to deliver fluid to, or withdraw fluid from, a brain of the subject.

Example Ex65 The method of Ex63, wherein the catheter is positioned to deliver fluid to, or withdraw fluid from, a CSF-containing space of the subject.

Example Ex66 The method of Ex64, wherein the catheter is positioned to deliver fluid to, or withdraw fluid from, a cerebral ventricle of the subject.

Example Ex67 The method of any one of Ex58 to Ex66, further comprising:

provoking the subject, wherein recording the electrical signal from the white or grey matter of the brain of the subject comprises recording a provoked electrical response.

Example Ex68 The method of Ex63, wherein provoking the subject comprises applying an electrical signal to the white or grey matter of the subject.

Example Ex69 A method comprising: (i) delivering fluid to, or withdrawing fluid from, a CSF-containing space of a brain of a subject, wherein the fluid is delivered or withdrawn through a first lumen of a CSF catheter; and (ii) recording an electrical signal from the white or grey matter via an electrode.

Example Ex70 The method of Ex69, wherein the electrode is fixed at a position relative to a distal end of the CSF catheter.

Example Ex71 The method of Ex69 or Ex70, wherein the CSF catheter comprises the electrode.

Example Ex72 The method of Ex69 or Ex70, wherein a lead comprises the electrode, and wherein the lead is secured relative to the CSF catheter.

Example Ex73 The method of any one of Ex69 to Ex72, wherein the catheter is positioned to deliver fluid to, or withdraw fluid from, a cerebral ventricle through the first lumen.

Example Ex74 The method of any one of Ex69 to Ex73, further comprising transmitting data regarding the recorded electrical signal to apparatus external to the subject.

Example Ex75 The method of any one of Ex69 to Ex74, further comprising determining a brain state of the subject or predicting a future brain state of the subject based on the recorded electrical signal.

Example Ex76 The method of any one of Ex69 to Ex75, further comprising provoking the subject, wherein recording the electrical signal from the white or grey matter of the brain of the subject comprises recording a provoked electrical response.

Example Ex77 The method of Ex76, wherein provoking the subject comprises applying an electrical signal to the brain of the subject.

Example Ex 78 A method according to Ex77, wherein the signal is applied by the electrode positioned in white or grey matter of the brain.

Example Ex79 A device or system comprising: (A) an implantable access port, the access port comprising: (ii) an opening accessible by a needle when the access port is implanted; (iii) a first catheter connector; (iv) a second catheter connector; (v) a first fluid flow path extending from the opening to the first catheter connector; and (vi) a second fluid flow path extending from the second catheter connector to the first catheter connector; (B) a CSF catheter coupled to, or operably couplable to, the first catheter connector, the CSF catheter comprising a proximal end, a distal end portion, and first and second lumens extending from the proximal end to the distal portion of the CSF catheter; and (C) an electrode, wherein the first lumen is in communication with the first fluid flow path and the second lumen is in communication with the second fluid flow path when the proximal end of the CSF catheter is coupled to the first catheter connector, wherein the CSF catheter has a length such that a distal end is configured to extend to a CSF-containing space of a subject when the access port is implanted, wherein the electrode is positioned a distance from the distal end of the CSF catheter such that the electrode is positioned in white or grey matter of a brain of the subject when the access port is implanted and the distal end of the CSF catheter is positioned in the CSF-containing space.

Example Ex80 The device or system of Ex79, wherein the second fluid flow path comprises a microbial filter.

Example Ex81 The device or system of Ex79 or Ex80, wherein the first fluid flow path is free of a microbial filter.

Example Ex82 The device or system of any one of Ex79 to Ex81, wherein the access port is configured to be implanted under a scalp of a subject.

Example Ex83 The device or system of any one of Ex79 to Ex82, wherein the CSF catheter comprises the electrode.

Example Ex84 The device or system of any one of Ex79 to Ex82, comprising a lead, wherein the lead comprises the electrode.

Example Ex85 The device or system of Ex84, wherein the lead is secured relative to the CSF catheter.

Example Ex86 The device or system of any one of Ex79 to Ex85, comprising signal apparatus electrically coupled to, or configured to electrically couple to, the electrode.

Example Ex87 The device or system of Ex86, wherein the signal apparatus is configured to be implanted under the scalp of the subject in proximity to an ear.

Example Ex88 The device or system of Ex86, wherein the CSF catheter comprises the signal apparatus.

Example Ex89 The device or system of Ex86, further comprising an implantable infusion device operably coupled to the second fluid flow path, wherein the implantable infusion device comprises the signal apparatus.

Example Ex90 The device or system according to any one of Ex86 to Ex89, wherein the signal apparatus is configured to process, transmit, or process and transmit data regarding electrical signals recorded by the electrode.

Example Ex91 The device or system of Ex90, further comprising external apparatus configured to receive data transmitted by the signal apparatus.

Example Ex92 The device of Ex91, wherein the external apparatus is configured to wirelessly power the signal apparatus.

Example Ex93 A method comprising: (A) providing a device or system comprising: (i) an implantable access port comprising: an opening accessible by a needle when the access port is implanted; a first catheter connector; a second catheter connector; a first fluid flow path extending from the opening to a first catheter connector; and a second fluid flow path extending from the second catheter connector to the first catheter connector; (ii) a CSF catheter coupled to, or operably couplable to, the first catheter connector, the CSF catheter comprising a proximal end, a distal end portion, first and second lumens extending from the proximal end to the distal portion of the CSF catheter, wherein the first lumen is in communication with the first fluid flow path and the second lumen is in communication with the second fluid flow path when the proximal end of the catheter is coupled to the first catheter connector, and wherein the CSF catheter has a length such that a distal end is configured to extend to a CSF-containing space of a subject when the access port is implanted; and (iii) an electrode positioned a distance from the distal end of the CSF catheter such that the electrode is positioned in white or grey matter of a brain of the subject when the access port is implanted and the distal end of the CSF catheter is positioned in the CSF-containing space; (B) implanting the device or system such that the proximal end of the CSF catheter is coupled to the first catheter connector, the first lumen of the catheter is in communication with the first fluid flow path of the access port, the second lumen of the CSF catheter is in communication with the second fluid flow path of the access port, the distal end of the CSF catheter is positioned in the CSF-containing space of the subject, and the electrode is positioned in the white or grey matter of the brain of the subject; and (C) recording an electrical signal from the white or grey matter via the one or more electrodes.

Example Ex94 The method of Ex93, wherein the access port is configured to be implanted under a scalp of the subject.

Example Ex95 The method of Ex93 or Ex94, wherein the CSF catheter comprises the electrode.

Example Ex96 The method of Ex93 or Ex94, wherein a lead comprises the electrode, and wherein the lead is secured relative to the CSF catheter.

Example Ex97 The method of any one of Ex93 to Ex96, wherein the distal end of the catheter is positioned in a cerebral ventricle.

Example Ex98 The method of any one of Ex93 to Ex97, comprising transmitting data regarding the recorded electrical signal to apparatus external to the subject.

Example Ex99 The method of any one of Ex93 to Ex98, comprising determining a brain state of the subject or predicting a future brain state of the subject based on the recorded electrical signal.

Example Ex100 The method of any one of Ex93 to Ex99, comprising provoking the subject, wherein recording the electrical signal from the white or grey matter of the brain of the subject comprises recording a provoked electrical response.

Example Ex101 The method of Ex100, wherein provoking the subject comprises applying an electrical signal to the brain of the subject.

Example Ex102 The method of Ex101, wherein the signal is applied by the electrode positioned in white matter or grey of the brain.

Example Ex103 The method of any one of Ex93 to Ex102, comprising infusing a therapeutic fluid through the second fluid flow path of the access port and through the second lumen of the catheter.

Example Ex104 The method of Ex103, wherein the second lumen is in communication with a cerebral ventricle.

Example Ex105 The method of Ex103 or Ex104, wherein the rate at which the therapeutic fluid is infused is altered based on data regarding the recorded electrical signal.

Example Ex106 The method of any one of Ex93 to Ex105, wherein the subject is at risk of or suffering from epilepsy, a head injury, stroke, subarachnoid hemorrhage, hydrocephalus, or brain infection.

Example Ex107 The method of Ex106, wherein the therapeutic fluid infused according to any one of Ex103 to Ex105 comprises an antiepileptic agent.

Example Ex108 The method of Ex107, wherein the antiepileptic agent comprises valproic acid or a pharmaceutically acceptable salt thereof.

Example 109 A method comprising: (i) implanting a catheter such that a distal end is positioned in a cerebrospinal fluid (CSF)-containing space of a brain of a subject, wherein implanting the catheter causes an electrode to be placed in contact with white matter or grey matter of the brain; (ii) infusing therapeutic fluid into the CSF-containing space, or withdrawing CSF from the CSF containing space, via a lumen of the catheter; and (iii) recording an electrical signal from the white or grey matter via the electrode.

Example Ex110 The method of Ex109, wherein the catheter comprises the electrode.

Example Ex111 The method of Ex109, wherein a lead comprises the electrode.

Example Ex112 The method of Ex111, comprising securing a portion of the lead to the catheter.

Example Ex113 The method of Ex111 or Ex112, comprising securing a distal portion of the lead to the catheter.

Example Ex114 The method of any one of Ex111 to Ex113, comprising securing a mid-portion of the lead to the catheter.

Example Ex115 The method of any one of Ex111 to Ex114, comprising securing a proximal portion of the lead to the catheter.

Example Ex116 The method of any one of Ex111 to Ex115, comprising securing the lead to the catheter along a substantial length of the catheter.

Example Ex117 The method of any one of Ex111 to Ex116, wherein securing the lead to the catheter comprises adhering the lead to the catheter.

Example Ex118 The method of any one of Ex111 to Ex117, wherein securing the lead to the catheter comprises mechanically fixing the lead to the catheter.

Example Ex119 The method of any one of Ex111 to Ex117, wherein securing the lead to the catheter comprises disposing a sleeve over the lead and the catheter.

Example Ex120 The method of Ex119, wherein the sleeve is elastomeric and configured to press the lead against the catheter when the sleeve is disposed over the lead and the catheter.

Example Ex121 The method of Ex119 or Ex120, wherein the sleeve comprises a window and the method comprises positioning the window over the electrode.

Example Ex122 The method of any one of Ex111 to Ex121, wherein the catheter defines a groove configured to receive the lead and wherein the method comprises disposing the lead in the groove.

Example Ex123 The method of Ex122, wherein the groove is configured to engage the lead via snap fit and the method comprises snaping the lead into the groove.

Example Ex124 The method of any one of Ex111 to Ex123, further comprising snapping the lead and the catheter into a snap fit connector.

Example Ex125 The method of any one of Ex109 to Ex124, wherein recording the electrical signal comprises recording an electrical signal from the white matter of the brain.

Example Ex126 The method of any one of Ex109 to Ex125, further comprising determining a brain state of the subject or predicting a future brain state of the subject based on the recorded electrical signal.

Example Ex127 The method according to any one of Ex109 to Ex126, further comprising: provoking the subject, wherein recording the electrical signal from the white or grey matter of the brain of the subject comprises recording a provoked electrical response.

Example Ex128 The method of Ex127, wherein provoking the subject comprises applying an electrical signal to the white or grey matter.

Example Ex129 The method of Ex128, wherein applying the electrical signal comprises applying the electrical signal via the electrode.

Example Ex130 The method of Ex128, wherein applying the electrical signal comprises applying an electrical signal via a stimulating electrode that is different than the electrode recording the electrical signal from the white or grey matter.

Example Ex131 The method of any one of Ex109 to Ex130, comprising positioning the distal end of the catheter in a cerebral ventricle.

Example Ex132 The method of Ex131, wherein the cerebral ventricle is a lateral ventricle.

Example Ex133 The method of any one of any one of Ex109 to Ex132, comprising infusing fluid into the CSF-containing space through a first lumen of the catheter, and withdrawing CSF from the CSF-containing space through a second lumen of the catheter.

Example Ex134 The method of any one of Ex109 to Ex133, wherein infusing fluid into the CSF-containing space comprises passing the fluid through a filter.

Example Ex135 The method of Ex134, wherein the filter is a microbial filter.

Example Ex136 The method of any one of Ex109 to Ex135, wherein withdrawing the fluid from the CSF-containing space comprises withdrawing the fluid without passing the fluid through a filter.

Example Ex137 The method of any one of Ex109 to Ex136, further comprising transmitting data regarding the recorded electrical signal to apparatus external to the subject.

Example Ex138 The device, system, or method of any one of Ex1 to Ex137, wherein the catheter (e.g., the cranial catheter) is an external ventricular drainage catheter or a ventriculoperitoneal shunt.

### EXAMPLES

Provided below are non-limiting examples that illustrate some aspects of the present disclosure.

### Example 1—Electrode Placement and Configuration

Electrode placement and configurations were evaluated to determine signal strength and signal to noise ratio. Differential and referential configurations were evaluated.

In differential mode, the system comprises an active electrode, a reference electrode, and a ground. The signal difference between an active electrode and a reference electrode may be amplified. The reference electrode may be a common reference for more than one active electrode. The reference electrode is preferably positioned a substantial distance from an active electrode and from the ground.

For a system as described herein, a differential mode recording may be achieved using four electrode (two electrodes per amplifier) and an additional ground electrode.

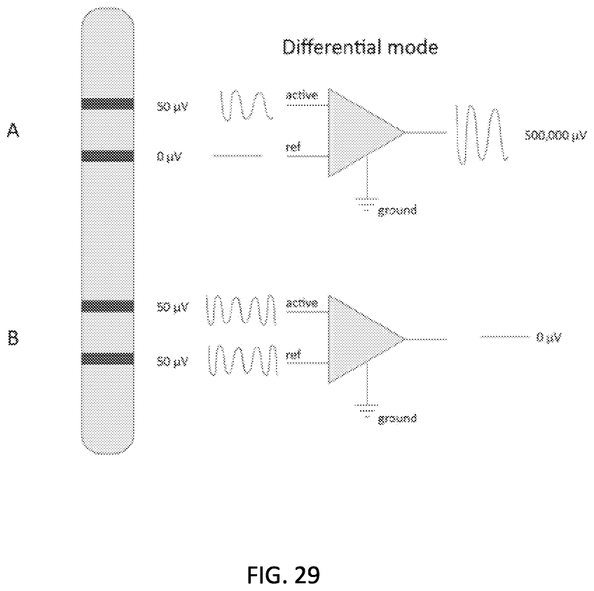

A schematic diagram illustrating signals in differential mode is shown in FIG. 29. In differential mode, the system may be configured to detect small differences between electrode pairs and may be less likely to be affected by large artifacts originating near the ground electrode (FIG. 29—A). However, the system may not be particularly effective at detecting larger common signals (FIG. 29—B).

To detect larger common signals, the system may be configured in referential mode, which may also be referred to as single-ended mode. Referential mode may use a single active electrode per amplifier. In referential mode, the output of the active electrode is amplified relative to the ground electrode, as opposed to the reference electrode in differential mode. The ground is preferably placed a substantial distance from the active electrode, which may result in amplification of signals that affect larger parts of the brain.

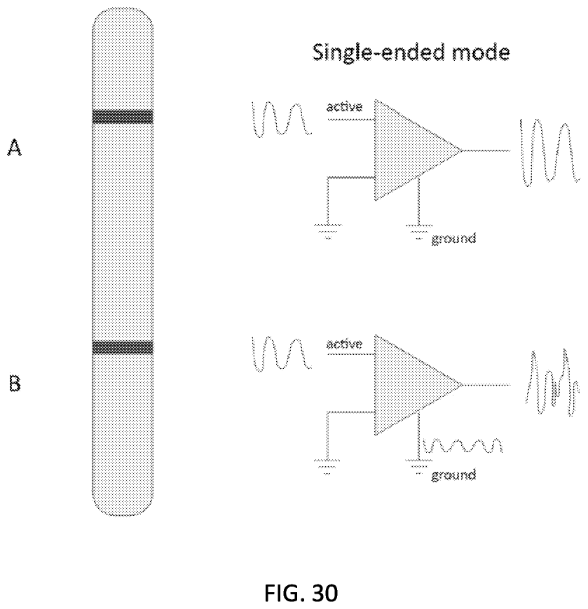

A schematic diagram illustrating signals in referential mode is shown in FIG. 30. While being effective at detecting larger common signals such as seizures (FIG. 30—A), referential mode may be sensitive to artifacts (FIG. 30—B). Proper placement of the ground electrode may mitigate some issues associated with artifacts.

To detect larger scale common signals, which may be more indicative of brain state, it is recommended to use referential mode recording while mitigating the effects of ground signal noise by careful consideration of electrode placement.

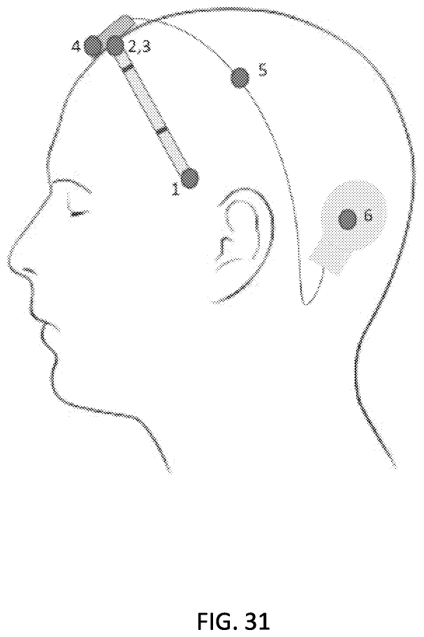

FIG. 31 is a schematic illustration of ground electrode placement that were considered. The six placements correspond to (1) on a CSF catheter at a distal end, (2) on the CSF catheter near an access port, (3) on the underside of the access port, (4) outside near the access port, (5) along a signal apparatus lead near a connector, and (6) on the signal apparatus behind the ear.

Table 1 compares the potential placement of the ground electrode and assigns a score (higher scores are considered better) for each of the locations relative to rejection of muscle artifact and movement, electrostatic and electromagnetic interference (ESD and EMC), recording area, signal level (amplitude), signal to noise ratio, and potential for large signal clipping (overload). Differential mode is shown for comparison.

TABLE 1
Scores for placement of ground
electrode-differential and referential mode

| Mode | EEG | Gn | G loc | Msc | Mvt | ESD | EMC | Reg | Sig | S/N | Over | Score |
|---|---|---|---|---|---|---|---|---|---|---|---|---|
| Diff | 4 | 0 | — | 5 | 5 | 5 | 5 | 1 | 1 | 1.0 | 5 | 32.0 |
| Ref | 2 | 1 | 1 | 5 | 5 | 5 | 5 | 2 | 2 | 2.0 | 5 | 39.0 |
| Ref | 2 | 1 | 2 | 5 | 5 | 4 | 4 | 4 | 5 | 4.7 | 4 | 53.0 |
| Ref | 2 | 1 | 3 | 3 | 5 | 1 | 1 | 4 | 4 | 2.4 | 3 | 36.2 |
| Ref | 2 | 1 | 4 | 3 | 3 | 1 | 1 | 5 | 3 | 1.4 | 1 | 31.2 |
| Ref | 2 | 1 | 5 | 2 | 3 | 1 | 1 | 5 | 3 | 1.2 | 1 | 29.6 |
| Ref | 2 | 1 | 6 | 2 | 4 | 1 | 1 | 5 | 4 | 1.9 | 1 | 33.6 |

| | | | | | | | | |
|---|---|---|---|---|---|---|---|---|
| Score Weights | 1 | 1 | 1 | 1 | 3 | 1 | 3 | 1 |

Mode: Diff (differential), Ref (referential), EEG: number of EEG electrodes, Gn: number of ground electrodes, G loc: location of ground electrode (see FIG. 31 and text above), Msc: Muscle artifact immunity, Mvt: electrode movement immunity, ESD: ESD immunity, EMC: EMC immunity, Reg: Recording region, Sig: Signal level, S/N: Signal-to-noise ratio, Over: Overload immunity, Score: Overall score.

Based on the information above, referential mode with the ground electrode placed on the CSF catheter (or on brain lead associated with the CSF catheter) near the access port (location 2 of FIG. 30) is recommended to detect larger scale changes, such as brain state.

### Example 2—Post-Traumatic Stress Disorder (PTSD)

The devices and systems disclosed herein may be used to treat or monitor any suitable disease. For purposes of illustration, treatment of PTSD and monitoring of electrical signals is discussed in more detail. A patient suffering from PTSD may be diagnosed according to criteria provided in DSM-5 (American Psychiatric Association. (2013). Diagnostic and statistical manual of mental disorders (5^{th} ed.). https://doi.org/10.1176/appi.books.9780890425596). The DSM-5 is incorporated herein by reference to the extent that it does not conflict with the disclosure presented herein.

Additional information regarding treatment and monitoring of PTSD and development of a PTSD DNN is described in U.S. Provisional Patent Application No. 63/166,705, filed on Mar. 26, 2021 and U.S. Provisional Patent Application No. 63/172,313, filed on Apr. 8, 2021, which applications are hereby incorporated herein in their respective entireties to the extent that they do not conflict with the disclosure presented herein.

High quality intracranial brain electroencephalography (iEEG) data may be obtained from PTSD patients employing the systems and devices described herein. The data may be used to better understand PTSD. The data may be used to monitor a state of the disease (such as severity), a state of the patient, or the like. The data may be used to direct, enhance, or direct and enhance PTSD therapy. The PTSD therapy may include one or more of cognitive therapy, psychological therapy, and pharmacological therapy.

Treatment of PTSD may include administration of a therapeutic agent us directly to CSF of a patient employing the devices and systems described herein. For example, the therapeutic agent may be administered to a cerebral ventricle. Administering to a cerebral ventricle enables delivery of the therapeutic agent adjacent to the specific region or regions of the brain responsible for severe traumatic memory recall. Such therapy may diminish the frequency, the severity, or the frequency and severity of PTSD intrusive memory recall from the hippocampus/basolateral amygdala memory neurons.

Any suitable therapeutic agent may be used to treat PTSD. For example, the therapeutic agent may be a selective serotonin reuptake inhibitor, an antihypertensive agent, or an antiepileptic agent. An example of a suitable antiepileptic agent is valproate or valproic acid. An example of a suitable form of valproate is sodium valproate.

The devices and systems described herein include catheters for delivering therapeutic agents to the CSF or delivering therapeutic agents to the CSF and for withdrawing CSF from the brain. A catheter may include one or more electrodes configured to be positioned within or close to white matter of the brain, grey matter of the brain, or white matter and grey matter of the brain. In addition or alternatively, a medical lead separate from a catheter may include one or more electrodes configured to be positioned within or close to white matter of the brain, grey matter of the brain, or white matter and grey matter of the brain. Preferably, the medical lead is configured to be coupled to, or is coupled to, a catheter so that implantation of the catheter results in implantation of the lead, if the catheter is implanted in the brain. That is, it is preferable that only one surgical procedure is performed to place the lead and the catheter. The electrodes may record electrical signals from within the brain.

By placing the electrodes within or in proximity to brain tissue, such as white matter or grey matter, less “noisy” signals may be obtained than with scalp-based EEG recordings. Such higher quality signals may facilitate interpretation of data recorded by the electrodes.

Preferably, the catheter comprises multiple lumens. A first lumen may be used, for example, to withdraw CSF from the brain, and a second lumen may be used, for example, to deliver a therapeutic fluid to the brain. The withdrawn CSF may be used to monitor disease or therapy progression, as well as monitor brain state, which may be used to, for example, adjust the rate of delivery of the therapeutic agent.

In addition or alternatively, data recorded by the electrodes may be used to monitor disease or therapy progression, as well as monitor a current brain state or predict a future brain state. The rate of delivery of the therapeutic fluid may be adjusted based on data recorded by the electrodes.

The combination of analysis of withdrawn CSF and data recorded by the electrodes may result in substantially improved therapy and therapeutic outcomes than analysis of either one alone.

Examples of studies that may be done are now provided.

### Overview of Potential Pilot Study

An implantable central nervous system (CNS) drug administration system and iEEG monitoring device may be implanted in 15 subjects with severe PTSD. In Aim 1, these subjects may receive a site specific therapeutic and may be included in the development of a novel iEEG PTSD focused database for symptom management which may be utilized for the development of a non-invasive PTSD diagnostic in the second aim.

In Aim 1, delivery of therapeutic through the minimally invasive central nervous system (CNS) drug administration system and iEEG monitoring device may be used to demonstrate initial clinical efficacy (improvement in the Clinician Administered PTSD Scale (CAPS-5) B Criterion, the standardized PTSD clinical questionnaire measure) and to collect PTSD specific implantable iEEG data.

In Aim 2, a PTSD DNN, trained on implanted iEEG data from the pilot clinical study, may be applied to a cohort of severe un-implanted PTSD subjects. Artificial intelligence (AI) may form the basis for iEEG analysis emphasizing those deep learning strategies initially shown most helpful for waking EEG classification for emotions, motor activity, cognitive activity, seizure detection and sleep scoring associated with an ongoing epilepsy trial. The course of sleep disturbances, a primary symptom of PTSD, may include ongoing analysis with local field potentials from deep brain electrodes subject to ongoing analysis by modern machine learning techniques as well as classical methods such as support vector machine and decision tree methods. Availability of both deep white matter and well as cortical recordings may facilitate control for brain state non-homogeneity during sleep states.

Significance and/or Uniqueness of the Proposed Effort

A primary significance of the work proposed resides in the development and utilization of novel treatment and unique brain data collection methods and, analytic strategies in subjects and novel treatments in a population with serious, treatment resistant PTSD. There are four major areas of significance in this realm:
1) EEG may be continuously recorded from locations within the brain (iEEG) while PTSD subjects reside at home going about routine life experiences. This may permit collection of brain as well as behavioral data including spontaneous symptom eruption obviating the artificial laboratory environment used in previous studies. Such recordings in a normal environment to identify the physiological signature of brain state changes accompanying a major mental disorder have never been previously possible outside of an artificial laboratory environment. The baseline recording period may permit establishment of normative values for sleeping, waking and changes associated with symptom onset.
2) PTSD symptomatic episodes, self-identified by subjects when they occur using, for example, a cell phone app, may be recognized immediately. EEG changes accompanying these PTSD symptomatic episodes as well as data preceding, accompanying, and following treatment may thus be isolated. Leveraging this critical data, a PTSD DNN may be developed using the latest deep learning techniques and AI strategies, with goals including identifying those specific brain state changes accompanying PTSD symptomatic episodes, as well as early brain state changes preceding and most probably predicting symptomatic episodes. A key goal is using an AI EEG DNN to reliably identify the specific neurophysiological altered PTSD brain states.
3) Following the baseline recording period, systematic treatment with ICV valproate may begin with individualized dose escalation until symptomatic episodes no longer occur or dose limiting side effects occur. This ICV treatment protocol is unique, with a goal of symptom control in a patient population previously non-responsive to treatment. Ongoing long term follow-up EEG recordings may determine possible resolution of previously identified waking and sleep abnormalities with restoration of normative values with effective treatment related symptom control, facilitating controlled dose reduction with ongoing physiological monitoring. This approach has never before been possible due to the inability to collect long term, high quality iEEG data. A second goal is initiating intraventricular drug delivery to minimize abnormal PTSD memory recall from the hippocampus.
4) Once EEG signatures described in (3) above have been obtained, AI analytic strategies may be utilized with surface EEG and other physiological measures (e.g. activity counts, heart rate and variability, skin conductance measures) with a goal of identifying non-invasive physiological measures capable of measuring and predicting PTSD related symptoms. Such measures have never been previously identified or described and represent a major scientific advance that could well be applied to other behavioral disturbances and their accompanying altered brain states, such as mood disorders and substance misuse, which also represent major problems for both military and civilian populations.

Site-Specific Hippocampal/Amygdala Drug Treatment for severe PTSD is novel and builds on work in minimally invasive epilepsy drug treatment. iEEG data is a novel application for PTSD, may expand on foundational non-invasive EEG and epilepsy work, and may facilitate unique novel clinical and product development strategy to further the targeted search for useful PTSD electrophysiologic biomarkers.

The combined PTSD approach offers the potential for patients with severe, non-remitting symptoms to receive treatment and resume normal life activities. An effective treatment and diagnostic may also benefit the family members who bear witness to the profound ongoing suffering and reduced quality of life of PTSD patients. A non-invasive diagnostic will not only enable triage and rapid diagnosis, but may resolve transparency issues, inform more invasive treatments, reduce PTSD stigma and prevalence, and improve outcomes for the full spectrum of PTSD sufferers.

Additional relevance of a reliable and effective treatment strategy is found in the diminution in adverse consequences and costs associated with untreated or inadequately treated PTSD in a number of biological domains. Depression and substance abuse are common comorbidities with PTSD, aggravated by continuation of PTSD symptoms. Chronic PTSD may be accompanied by increases in inflammatory markers including interleukin 6, interleukin 1β, TNFα, and interferon γ levels, supporting low grade inflammation with adverse pathophysiological implications including decreased neurogenesis leading to brain volume loss, impairment in memory formation and consolidation, and acceleration of aging via reactive oxygen species mediated exacerbation of telomere dysfunction and cell senescence, which could explain the association between PTSD and aging. PTSD is also accompanied by increases in REM sleep without atonia, a harbinger of REM behavior disorder with its associated increases in risk for future neurodegenerative disease.

Another PTSD related issue relates to risk factors for development of symptomatic PTSD in response to stress. In fact, only a minority of severely stressed individuals develop PTSD, approximately 20-30% being worse case. The other 70-80% of individuals do not develop PTSD after experiencing similar stress. Identification of those at risk beforehand would be very helpful in terms of decreasing incidence of PTSD and associated social, medical, and military costs. Two major risk factors for development of PTSD in response to stress are socioeconomic status (SES), and early childhood experience. The risk for developing PTSD in recent military veterans is higher than that of the civilian population following major traumatic stress, and SES is a major factor separating military from civilian populations.

The enhanced treatment approach has the potential to enhance recovery rates and quality of life patients suffering from PTSD.

The development of such new strategies for identification and modulation of brain states accompanying other significant behavioral disorders such as mood disorders, anxiety disorders, problems associated with substance misuse and related issues may have wide ranging impact in terms of early identification and treatment before disorders become chronic and disabling.

This PTSD study may evaluate if the test drug and devices are safe and effective for the study population and if the iEEG recordings may be useful with AI in identifying and detecting key biomarkers for the core symptoms of PTSD. The primary goal of this preliminary study is to evaluate if the intervention shows promise of efficacy for patients with PTSD. A positive outcome would provide evidence of lessened distress related to traumatic memory recall. Patients may experience a reduction in the frequency of intrusive and aversive memory recall, in the distress produced by aversive memory recall, or reduced persistence of such episodes of aversive memory recall.

Another goal of this preliminary study is to examine the impact of the intervention on sleep phenomenology related to PTSD. A positive outcome may provide evidence that the intervention is associated with reduced frequency of disrupted sleep, reduced frequency and/or severity of nightmares, and reduced impact of nightmares on daily function. In addition, this preliminary study may examine whether the severity of other chronic comorbid conditions is altered after starting the intervention, including symptoms of anxiety and depression.

This study also aims to use an AI PTSD DNN to identify EEG biomarkers of core PTSD symptoms (spontaneous traumatic memory recall, frequency of recall, level of distress, sleep symptoms). In particular, the goal is to determine whether these EEG biomarkers provide a sensitive and specific objective index of the therapeutic impact of the intervention. A related goal is to examine the timing of changes in potential EEG biomarkers in relation to changes in physical activity and heart rate. Specifically, the goal will be to test the predictive value of changes in these measures for the onset of new episodes of aversive memory recall. This information may prove valuable in designing early interventions that may prevent subsequent flashback/panic episodes and reduce the burden of PTSD overall.

After providing informed consent, patients may be screened for inclusion/exclusion criteria and all patients may continue their prescribed, orally-administered PTSD and other concomitant medications throughout the trial. Clinical assessments, adverse events, PTSD diary, concomitant medications, blood samples and cerebral spinal fluid will be collected and reviewed at designated time points. MRI Scan and ECG may also be performed at baseline and study completion. Subjects may have the surgery and any dose changes and pharmacokinetic sampling performed in an inpatient setting.

After completion of the surgery implant, subjects may participate in a 4-6 month baseline during which the focus will be on collecting behavioral, EEG and other physiological data which may characterize their mood states (electrophysiologic data and autocurate rating of their mood state) and identify episodes of spontaneous PTSD memory recalls including depersonalization, overwhelming dread associated with those recalls, and nightmares.

Over the next month, the ICV Valproate dose may be introduced and escalated over four weeks, if tolerated, or stopped earlier upon establishment of a subject's maximum tolerated dose (MTD). After establishing a patient's MTD, delivery of ICV Valproate may continue at the MTD for three months during the dosing Evaluation Period and compared with baseline.

During each week of the dose escalation, during the Primary Evaluation Period and Long-Term Follow-up, patients may have 24 hours a day EEG recording which includes sleep stage, circadian and ultradian rhythm calculations along with activity monitoring, heart rate and variability. Psychometric assessments will be performed on a scheduled basis.

A high-quality EEG database on PTSD patients analyzable using cutting edge Artificial Intelligence (AI), may be important to making diagnostic advancement in this field. The long-term EEG data for such database and AI models may only be accessible through invasive approaches, which is feasible and potentially beneficial to severe patients when the collection of this data is coupled with a low-risk therapeutic drug infusion.

This PTSD approach builds on Cerebral Therapeutics, Inc.'s active drug (reformulated ICV valproate, CT 010) and catheter development (ICVRx) programs for severe epilepsy, a long-term brain iEEG epilepsy data set, and the documented success of more aggressive therapies in subsets of severe psychiatric patients. High-quality brain iEEG data collected while providing site-specific medication treatment of severe PTSD patients may be foundational toward building a non-invasive AI based predictive diagnostic. The non-invasive PTSD diagnostic may function on lower-quality non-invasive data from severe PTSD patients, later expanding to non-invasive data sets for diagnosis of mild and moderate PTSD patients.

### Prophetic Protocol

The following prophetic example provides a summary of a trial protocol that may be employed to evaluate the principles described herein.

Test drug: Cerebral Therapeutics—Sodium Valproate for ICV use 90 mg/ml (CT 010)

Test Infusion pump: Tricumed—IP 2000V Implantable Pump

Catheter/access port: Cerebral Therapeutics—ICVRx Smart Implantable ICV Catheter

Study Indication: Treatment-resistant PTSD

Synopsis: Evaluate if the Test Drug (CT 010) and Devices (IP 2000V and ICVRX Smart) are safe and effective for the Study Population and if the ICVRx Smart Catheter will be useful in identifying and detecting key biomarkers for the core symptoms of PTSD. The study will first have an enrollment period when screening is completed, followed by device implantation, followed by a PTSD symptom monitoring period, followed by Investigational Product dose escalation, followed by the primary evaluation period which is followed by the Long-Term Follow up.

Patient population inclusion criteria: Female or Male patients between age 18-70 that are diagnosed with PTSD as defined by the Diagnostic and Statistical Manual fifth edition (DSM-5). Treatment resistance may be defined by the persistence of at least moderate clinical symptoms despite adequate treatment (dose, duration and compliance) with three modalities, including a) selective serotonin reuptake inhibitors, b) cognitive behavioral therapy, c) other classes of medications and/or psychotherapy. Patients should exhibit moderate-to-severe forms of the disease as measured by Clinician Administered PTSD Scale (CAPS) scores ≥50. The PTSD should be chronic, non-remitting PTSD with duration >2 years. Patients should have the capacity to provide informed consent and comply with all evaluations and protocols and ability to attend study appointments. Patients should meet inclusion criteria for at least one of symptoms listed below. All symptoms need not be present for enrollment: (i) Unwanted memories occurring >20 times per month at enrollment baseline; (ii) Dissociative Reactions Flashbacks occurring >3 times per month at enrollment baseline.

Patient population exclusion criteria: (i) patients with past or current psychosis or mania; (ii) history of schizophrenia, schizoaffective disorder, psychotic depression or bipolar 1 disorder; (iii) history of at most mild Traumatic Brain Injury (Physician evaluation, VA Traumatic Brain Injury Screening Tool (VATBIST) 198), (iv) active neurologic disease including epilepsy, Parkinson's disease, multiple sclerosis, Alzheimer's disease, other dementias, etc.; (v) alcohol or substance dependence or abuse in the last 6 months, excluding caffeine and nicotine; (vi) active suicidal intent or suicide attempt within the last 2 years; (vii) contraindication to CT or MRI; (viii) likely to relocate or move out of the country during the study's 3 year duration; (ix) presence of a neurological or other medical condition that significantly increases the risk of the surgical procedure; (x) currently pregnant (as determined by history and serum HCG) or lactating; for females of reproductive potential: use of highly effective contraception for at least 1 month prior to screening and agreement to use such a method during study participation; (xi) obstruct sleep apnea-(Initial study exclude with AAHI >10 or 15).

Study goal: (i) A primary goal of is to evaluate if the intervention shows promise of efficacy for patients with PTSD. A positive outcome would provide evidence of lessened distress related to traumatic memory recall. Patients may experience a reduction in the frequency of intrusive and aversive memory recall, in the distress produced by aversive memory recall, or reduced persistence of such episodes of aversive memory recall. (ii) Another goal is to examine impact of the intervention on sleep phenomenology related to PTSD. A positive outcome would provide evidence that the intervention is associated with reduced frequency of disrupted sleep, reduced frequency and/or severity of nightmares, and reduced impact of nightmares on daily function. In addition, this preliminary study will examine whether the severity of other chronic co-morbid conditions is altered after starting the intervention, including symptoms of anxiety and depression. (iii) In addition, the study may identify EEG biomarkers of core PTSD symptoms (spontaneous traumatic memory recall, frequency of recall, level of distress, sleep symptoms, etc.). In particular, the goal is to determine whether the EEG biomarker provides a sensitive and specific objective index of the therapeutic impact of the intervention. (iv) A related goal is to examine the timing of changes in potential EEG biomarkers in conjunction in relation to changes in physical activity and heart rate. Specifically, the goal will be to test the predictive value of changes in these measures for the onset of new episodes of aversive memory recall. This information may prove valuable in designing early interventions that may prevent subsequent flashback/panic episodes and reduce the burden of PTSD overall.

Study design: (i) After providing informed consent, patients may be screened for inclusion/exclusion criteria and all patients may continue their prescribed, orally administered PTSD and other concomitant medications throughout the trial. Clinical assessments, AEs, PTSD diary, concomitant medications, blood samples and CSF may be collected and reviewed at designated time points. MRI Scan, and ECG will also be performed at baseline and study completion. (ii) subjects may have the surgery, any dose change and pharmacokinetic sampling performed in an inpatient setting. (iii) After completion of the surgery implant, subjects may participate in a four-to-six-month baseline during which the focus will be on collecting behavioral and EEG and other physiological data which may characterize their mood states (electrophysiologic data and autocurate rating of their mood state) and identify episodes of spontaneous PTSD memory recalls including depersonalization, overwhelming dread associated with those recalls, and nightmares. (iv) Over the next month, the ICV Valproate dose may be introduced and escalated over four weeks (50, 100, 150 and 200 mgs/day of ICV Valproate), if tolerated, or stopped earlier upon establishment of a subject's maximum tolerated dose (MTD). The MTD for each patient may be determined based on the highest dose tolerated without experiencing a dose-limiting AE. After establishing a patient's MTD, delivery of ICV Valproate may continue at the MTD for three months during the dosing Evaluation Period and compared with baseline. (v) Continuously, during each week of the dose escalation, during the PEP (Primary Evaluation Period) and LTFU (Long Term Follow-up), patients may have 24 hours a day EEG recording which includes sleep stage, circadian and ultradian rhythm calculations along with activity monitoring, heart rate and variability. Psychometric assessments may be performed on a scheduled basis and may include CAPS, BDI, BAI Columbia-Suicide Severity Rating Scale (C-SSR) (McCall) (scale for active suicide ideation), and PSQI (Pittsburgh Sleep Quality Index). (vi) As patients may have failed multiple meds and therapy, patients may have medication changes limited and documented during the study at the treating physician and medical monitor discretion to limit patient's severe symptom exacerbation.

Objectives (endpoints): (i) Evaluation of therapy related Serious Adverse Events and Unanticipated Adverse Device Effects related to procedure, drug and/or device. (To be determined: SAFTEE, UKU, MADRS). (ii) Reduction in CAPS PTSD Score that is meaningful (by 30-50% PTSD) CAPS B1 (Memories), B2 (Daytime Flashbacks) and B3 (Nightmares) sub score particularly to improvement. Need to decide one and three months. (iii) Global Secondary Endpoints and Comorbidity: (a) Difference in cognition, (b) Patient Global Impression of Change (PGIC) (1-7, high better), (c) Clinical Global Impact of Change (CGIC) (1-7, low better), (d) Patient Health Questionnaire 9-item Depression scale (PHQ-9), (0-27), (e) Quality of Life Inventory (QOLI), (−6 to 6), (f) PCL-5 20 item self-report (20 DSM-5 symptoms of PTSD), (g) Beck Depression and Anxiety Inventories (BDII-II; BAI) 20 item self; (iv) Sleep Secondary Endpoints: (a) Improvement in objective sleep markers including: (1) N3 Slow wave Delta sleep (quantitate), Total REM sleep (quantitate), (2) Change in sleep spindle frequency (Wang2020), Sleep total Sleep Time (TST), Increased Sleep Efficiency (SE), Sleep Latency (SL), Increased Total Sleep Times, and Sleep spectral changes (including increased delta power suggesting increased homeostatic sleep drive, decreased beta/gamma power suggesting decreased hyperarousal state) (Wang2020 #2), (3) Pittsburgh Sleep Quality Index (PSQI), 0-21), and (4) Decreased REM interruptions (Habakawau); (v) EEG BioMarkers Comparison at differing dose levels & versus baseline including: (a) Non AI Gamma Band, (b) P300; (v) AI Features

### Example 3—Traumatic Brain Injury

Traumatic brain injury (TBI) remains a leading cause of disability and death in children and adults in their most productive years. It is also a leading cause of morbidity and mortality in the military. Every year an estimated 1.6 million head injuries occur in the US. TBI affects three out of every 1,000 Americans annually, accounting for as many as 60,000 deaths and an estimated 70,000 to 90,000 individuals with chronic neurological disabilities. The economic consequences of these injuries are enormous in terms of lost productivity and medical care costs. The direct and indirect costs of TBI in the US are estimated to be $48.3 billion annually. Survivor costs account for $31.7 billion and fatal brain injuries amount to another $16.6 billion. While the last two decades of research has resulted in a greater understanding of the physiological and cellular events leading to secondary neuronal injury, and methods to characterize changes in cerebral blood flow and intracranial pressure have modestly improved, there remains a profound lack of brain functional measurement tools starting at the time of acute injury through rehabilitation which provide objective metrics to guide treatment decisions in patients with severe TBI.

Management of severe TBI and associated elevated intracranial pressure (ICP) typically necessitates placement of an externalized ventricular drain (EVD). This implant accesses the ventricle via a flexible tube and drains cerebral spinal fluid (CSF) to lower ICP. This procedure, common in neurosurgical practice, is a cornerstone of evidence-based care for patients presenting with severe traumatic closed head injury. Each year, approximately 23,000 EVDs are placed in the US and 4,000 (or 17%) are placed for presumed severe closed head injury.

Patients with EVDs typically go through a two-week period of intensive care during which the patient is often intubated and aggressive multisystem care is focused on preserving life and brain function. These patients typically remain in the hospital for another two or so while caregivers attempt to find placement in a long-term or rehabilitation facility. Challenging behavioral presentations are nearly universal, as is use of psychotropic medications to manage them. A common course is for patients to move from an inpatient setting into at least a year of nursing care or rehabilitation, depending on the level of consciousness, cognition, language, and behavioral disturbance. Despite the obvious criticality of the evolving brain injury, physicians lack objective data regarding brain physiology and function, relying instead on clinical observation and, when possible, self-report to inform treatment decisions. Beginning with the acute onset of injury through the first year of recovery, the clinical challenges evolve, starting with the elevated intracranial pressure, extending to sleep disturbance in the ICU, additional behavioral and sleep challenges in the post-extubation hospitalization period, and later focus on the cognitive, impulse control, and behavioral challenges that interfere with occupational, speech, and physical therapy that, nonetheless, are key to optimizing brain outcomes over the first year

To date, while EVDs are surgically implanted in thousands of patients with TBI, they have been employed and designed to date solely to lessen ICP. Current EVD devices provide no information about brain physiology and function despite the great need for objective biomarkers of evolving brain states and despite the fact that EVD devices are already being located physically implanted in patients' brains.

Monitoring of electrical brain signals may include obtaining high quality intracranial brain electroencephalography (iEEG) data from TBI patients. The data may be used to better understand TBI. The data may be used to monitor a state of the disease (such as severity), a state of the patient, or the like. The data may be used to direct, enhance, or direct and enhance TBI therapy. The TBI therapy may include one or more of cognitive therapy, psychological therapy, pharmacological therapy, and ventricular drainage.

CSF may be drained through the use of a drainage catheter having an opening placed in a cerebral ventricle to an opening located outside of the CSF space, such as external to the patient. CSF drainage may reduce ICP.

The CSF drainage catheter may contains one or more electrodes for recording signals from the patient's brain. One or more electrodes of the drainage catheter may be configured to be positioned within or close to white matter of the brain, grey matter of the brain, or white matter and grey matter of the brain. In addition or alternatively, a medical lead separate from a drainage catheter may include one or more electrodes configured to be positioned within or close to white matter of the brain, grey matter of the brain, or white matter and grey matter of the brain. Preferably, the medical lead is configured to be coupled to, or is coupled to, a drainage catheter so that implantation of the drainage catheter results in implantation of the lead, if the drainage catheter is implanted in the brain. That is, it is preferable that only one surgical procedure is performed to place the lead and the drainage catheter. The electrodes may record electrical signals from within the brain.

By placing the electrodes within or in proximity to brain tissue, such as white matter or grey matter, less “noisy” signals may be obtained than with scalp-based EEG recordings. Such higher quality signals may facilitate interpretation of data recorded by the electrodes.

Data recorded by the electrodes may be used to monitor disease or therapy progression, as well as monitor a current brain state or predict a future brain state. Electrical biomarkers may be identified. Such objective biomarkers of disturbed brain function in TBI can provide both targets for therapeutic interventions and valid metrics by which to evaluate the impact of modifications and tailoring of current available treatment strategies. This is especially important because the neuropsychiatric disturbances in TBI frequently compromise the reliability and validity of self-report. Treatment strategies guided by objective biomarkers may result in superior outcomes and shorter treatment periods in intensive care units, hospital, and rehabilitation settings.

Additional information regarding treatment and monitoring of TBI and development of a TBI DNN is described in U.S. Provisional Patent Application No. 63/223,629, filed on Jul. 20, 2021, which application is hereby incorporated herein in its entirety to the extent that it does not conflict with the disclosure presented herein.

Examples of studies that may be done are now provided.

### Overview of Potential Pilot Study

Collection of high quality, continuous iEEG data may occur during clinician management of TBI. A first step may be focus on epileptiform and sleep stage detection (high risk and frequent complications for these patients). A second may be to expand the data scope applying labeling strategies with the continuous iEEG during the course of their recovery to identify patterns of brain electrical activity that reflect abnormal brain states and/or covary with behavioral disturbance.

In initial phases after injury, detection of undiagnosed epileptiform conditions and sleep disorders may be prioritized using conventional techniques combined with AI methods. As is demonstrated in an epilepsy iEEG pilot data section, advanced AI-DNN methods may subsequently be applied to drive the identification of potential biomarkers, emphasizing and building on application of those deep learning strategies shown most helpful for EEG classification of sleep stages and epileptiform activity. This approach may be expanded from sleep and epileptiform activity to iEEG-DNNs linked to specific disturbances in emotion, motor activity, or cognition. The characterization of sleep disturbances, which may evolve with closed head injury, may include longitudinal analysis of local field potentials from deep brain electrodes using modern machine learning techniques, as well as classical methods, such as support vector machine and decision tree methods. The availability of recordings from both cortical and deep white matter regions may facilitate control for brain state non-homogeneity during sleep states.

During the subsequent phases of recovery, in addition to providing iEEG objective biomarkers of sleep and epileptiform disturbance, it is postulated that the iEEG-DNN approach may provide objective biomarkers useful in the diagnosis and ultimately the management of behavioral disturbances, such as agitation, inattention, or disorientation. Behavioral disturbance in the TBI patient are often incompletely or poorly controlled with psychotropics. Since the iEEG can be continuously collected and because the iEEG implant can remain in the patient even after the EVD has been removed, the TBI DNN strategy can then be expanded over the longer course of recovery to explore the potential of biomarker identification for a variety of neuropsychiatric complications, including sleep disturbances, agitation, PTSD, suicidal and impulsive behaviors.

Detecting EEG sleep and epileptiform activity patterns using an extremely more capable iEEG collecting implant may enable more accurate and clinically meaningful assessment of change in these domains. Given our knowledge of sleep architecture and the nature of epileptiform activity, disturbances in these domains are likely analyzable based on iEEG analysis alone. However, the development of TBI iEEG DNNs may be important for many brain dysfunctions, such as agitation, suicidality, or attentional dysfunction which may require linkage between the brain data and behavioral manifestations. Tagging strategies may be used for such linkage by an experienced AI data analytic and specific PTSD psychiatry team.

Cerebral Therapeutics, Inc. has previously analyzed a pilot data set from a unique epilepsy cohort with just this type of data analytic approach and was able to recognize sleep stages and epileptiform discharges comparable with the proposed modified iEEG ventriculostomy.

In Aim 1, a minimally invasive ventriculostomy catheter and iEEG monitoring device may be used in a pilot study of 20 severe TBI participants to demonstrate potential clinical utility (effective CSF drainage and quality sleep and EEG data captured as compared with scalp EEG data capture). In Aim 1, specific iEEG data signatures for epileptiform activity and sleep disturbance will be generated. The iEEG device acquisition system may record from 2× electrodes distributed over a intracranial depth of approximately 4× cm, with the signals externally digitized (250 Hz sampling rate). This arrangement may produce approximately 0.095 Gigabyte of data per day per patient (about 3 GB per month per patient). The internalized system may be compatible with routine use of ventriculostomy and externalized iEEG capture and digitalization technology may be wearable, and ergonomically and unobtrusively designed, to support cloud-based data transfer.

In Aim 2, the pilot study findings may be expanded to include preliminary application of TBI DNN strategy to the domains of agitation, cognitive impairment, and, when appropriate, PTSD or suicidal ideation. Just as innovation and advanced analytic techniques may be applied to the iEEG data, state-of-the-art methods may be applied when “labelling” contemporaneous behavioral events. The behavior collection strategies may include analysis of video recordings, nursing and caregiver daily reports, medications and their administration times, continuous recording of ongoing physiology (e.g., heart rate, respiration, etc.) and, when feasible, self-report of subjective states and objective task performance.

After successfully addressing Aims 1-2, the follow-on stages of development of the may proceed, progressing towards commercialization and full regulatory (e.g., FDA) clearance. Specifically, this may mean conducting an appropriately powered and controlled clinical trial demonstrating the clinical utility and safety of the technology and iEEG analyses. The design and implementation of a current Phase 2b epilepsy trial conducted by Cerebral Therapeutics, Inc. may be helpful to model an approach for the proposed TBI patient investigation.

A primary significance of the proposed research resides in the collection and utilization of unique brain data and novel analytic strategies to develop biomarkers in a population with severe TBI. There are four major areas of significance in this realm:
1) The collection of high-quality brain data, built upon solid experience with acquisition and analysis of intraventricular sleep and epilepsy continuous iEEG, has never been routinely obtained in patients with severe brain injury, either acutely or over the long-term over the course of their brain injury and recovery. Acquiring these key data provides an unparalleled opportunity for the field: unique high-quality, in the brain, objective data by which to investigate the optimal delivery of rehabilitation interventions and prescriptions (i.e., frequency, intensity, timing, and type), as well as to investigate the comparative effectiveness of standard of care and novel intervention strategies. The functional brain data gap is not unique among brain diseases to severe TBI. However, what is unparalleled is the opportunity to access and analyze these unique brain data with minimal or no added risk and tremendous possibility of gain for patients and the field. Thousands of such patients have EVDs implanted to reduce ICP. Yet, these EVDs currently provide no information about brain physiology or function.
2) During the acute and first weeks of treatment, the continuous iEEG data may be used to monitor for epileptiform activity and sleep disruption. Sleep disturbance and epileptiform activity following TBI are well known to disrupt recovery, impacting on symptom manifestation and behavioral changes, and the management of sleep disturbance and epileptiform activity can be improved upon in many severe TBI patients. Once EEG signatures described in (1) above have been obtained, AI DNN analytic strategies may be utilized and compared with surface EEG and other physiological measures (e.g., activity counts, heart rate and variability, skin conductance measures and others) to initially test sensitivity and specificity in detecting and predicting sleep disturbances and indicators of seizure activity. Based on the much higher quality data, it is expected that the biomarkers identified with ciEEG and AI DNN detection may demonstrate stronger sensitivity and specificity than scalp EEG.
3) After the first 4-6 weeks phase, the iEEG data may continue to be collected, along with additional monitoring for attentional challenges and anxiety symptoms which may trigger oral medication intervention to help patients improve their post-injury course. Similar iEEG and DNN AI techniques and strategies may be applied as in Aim 1, but at this point with tagging to specific behavioral states (e.g., agitation) or subjective states (PTSD, suicidality).
4) Objective biomarkers in TBI of abnormal brain states and behavioral disturbances have never been previously identified and could radically alter clinical management by providing objective and outcome relevant endpoints. This data-driven, empirical strategy represents a major scientific advance that could be applied to other forms of behavioral disturbance and their accompanying altered brain states, such as mood disorders and substance misuse, which also represent major problems for both military and civilian populations.

What is claimed is:

1. A device or system for delivering fluid to or removing fluid from a cerebrospinal fluid (CSF)-containing space of a brain and for recording electrical activity from white or grey matter in the brain, the device or system comprising:
a CSF catheter comprising a proximal end, a distal end portion, and a first lumen extending from the proximal end to the distal end portion; and
an electrode positioned a distance from a distal end of the CSF catheter such that the electrode would be placed in contact with white or grey matter of the brain if the distal end of the CSF catheter were positioned in the CSF-containing space.

2. The device or system of claim 1, wherein the CSF-containing space is a lateral ventricle.

3. The device or system of claim 2, wherein the electrode is positioned a distance from about 0.5 centimeters to about 6 centimeters to the distal end of the CSF catheter.

4. The device or system of claim 2, wherein the electrode is positioned a distance from about 1 centimeter to about 5 centimeters to the distal end of the CSF catheter.

5. The device or system of claim 2, wherein the electrode is positioned a distance from about 2 centimeters to about 6 centimeters to the distal end of the CSF catheter.

6. The device or system of claim 1, wherein the CSF catheter comprises the electrode.

7. The device or system of claim 1, further comprising a brain lead, wherein the brain lead comprises the electrode and wherein the brain lead is coupled to the CSF catheter.

8. The device or system of claim 7, wherein the brain lead is coupled relative to the CSF catheter at the distal end portion of the CSF catheter.

9. The device or system of claim 7, wherein the brain lead is secured to the CSF catheter along a substantial length of the CSF catheter.

10. The device or system of claim 1, comprising a brain signal electrical interconnect, wherein the brain signal electrical interconnect comprises a contact electrically coupled to the electrode.

11. The device or system of claim 10, wherein the CSF catheter comprises the brain signal electrical interconnect.

12. The device or system of claim 10, comprising the brain lead according to claim 7, wherein the brain lead comprises the brain signal electrical interconnect.

13. The device or system of claim 10, wherein the CSF catheter comprises the electrode and wherein the brain signal electrical interconnect is physically separate from the CSF catheter.

14. The device or system of claim 13, further comprising a cable extending from the CSF catheter to the brain signal electrical interconnect, wherein the cable comprises a conductor electrically connecting the electrode to the contact.

15. The device or system of claim 1, further comprising an access port having a first fluid flow path in communication with the first lumen of the CSF catheter or having a connector configured to secure the CSF catheter such that first lumen is in communication with the first fluid flow path.

16. The device or system of claim 15, wherein the access port is configured to be implanted below a scalp.

17. The device or system of claim 16, wherein at least a portion of the access port is configured to be implanted over a burr hole.

18. The device or system of claim 16, wherein at least a portion of the access port is configured to be implanted in the burr hole.

19. The device or system of claim 1, further comprising signal apparatus for processing, transmitting, or processing and transmitting data regarding the signals recorded by the electrode.

20. The device or system of claim 19, further comprising an implantable infusion device, wherein the implantable infusion device comprises the signal apparatus.

21. The device or system of claim 1, wherein the cranial catheter is an external ventricular drainage catheter or a ventriculoperitoneal shunt.

22. A device or system comprising:
an implantable access port, the access port comprising:
an opening accessible by a needle when the access port is implanted;
a first catheter connector;
a second catheter connector;
a first fluid flow path extending from the opening to the first catheter connector; and
a second fluid flow path extending from the second catheter connector to the first catheter connector;
a CSF catheter coupled to, or operably couplable to, the first catheter connector,
the CSF catheter comprising a proximal end, a distal end portion, and first and second lumens extending from the proximal end to the distal portion of the CSF catheter; and
an electrode,
wherein the first lumen is in communication with the first fluid flow path and the second lumen is in communication with the second fluid flow path when the proximal end of the CSF catheter is coupled to the first catheter connector,
wherein the CSF catheter has a length such that a distal end is configured to extend to a CSF-containing space of a subject when the access port is implanted,
wherein the electrode is positioned a distance from the distal end of the CSF catheter such that the electrode is positioned in white or grey matter of a brain of the subject when the access port is implanted and the distal end of the CSF catheter is positioned in the CSF-containing space.
